SEQUENCE LISTING (2 sequences)

SEQ ID NO: 1 - DNA, 40 bp, Artificial Sequence
  Description of Artificial Sequencetail for forward primer for yeast two-hybrid system
gcaggaaacagctatgaccatacagtcagcggccgccacc

SEQ ID NO: 2 - DNA, 39 bp, Artificial Sequence
  Description of Artificial Sequencetail for reverse primer for yeast two-hybrid system
acggccagtcgcgtggagtgttatgtcatgcggccgcta

(19) United States
(12) Patent Application Publication
(10) Pub. No.: US 2002/0048769 A1
(43) Pub. Date: Apr. 25, 2002

(54) Protein-protein interactions in neurodegenerative disorders

(75) Inventors: Jean-Marc Roch, Salt Lake City, UT (US); Paul L. Bartel, Salt Lake City, UT (US)

Correspondence Address: ROTHWELL, FIGG, ERNST & MANBECK, P.C., 555 13TH STREET, N.W., SUITE 701, EAST TOWER, WASHINGTON, DC, 20004, US

(73) Assignee: MYRIAD GENETICS, INC., Salt Lake City, UT

(21) Appl. No.: 09/970,814

(22) Filed: Oct. 5, 2001

Related U.S. Application Data
(62) Division of application No. 09/466,139, filed on Dec. 21, 1999.
(60) Provisional application No. 60/113,534, filed on Dec. 22, 1998.
(60) Provisional application No. 60/124,120, filed on Mar. 12, 1999.
(60) Provisional application No. 60/141,243, filed on Jun. 30, 1999.

(51) Int. Cl. C12Q 1/68; G01N 33/53; C12N 9/16; C07K 16/42

(52) U.S. Cl. 435/6; 435/7.1; 435/196; 530/388.1

(57) ABSTRACT

The present invention relates to the discovery of protein-protein interactions that are involved in the pathogenesis of neurodegenerative disorders, including Alzheimer's disease (AD). Thus, the present invention is directed to complexes of these proteins and/or their fragments, antibodies to the complexes, diagnosis of neurodegenerative disorders (including diagnosis of a predisposition to and diagnosis of the existence of the disorder), drug screening for agents which modulate the interaction of proteins described herein, and identification of additional proteins in the pathway common to the proteins described herein.

### CROSS-REFERENCE TO RELATED APPLICATION

The present application is a divisional application of U.S. patent application Ser. No. 09/466,139, filed Dec. 21, 1999. The present application is related to U.S. provisional patent application Ser. No. 60/113,534, filed Dec. 22, 1998, to U.S. provisional patent application Ser. No. 60/124,120, filed Mar. 12, 1999, and to U.S. provisional patent application Ser. No. 60/141,243, filed Jun. 30, 1999, each of which are incorporated herein by reference, and claims priority thereto under 35 USC § 119(e).

### BACKGROUND OF THE INVENTION

The present invention relates to the discovery of protein-protein interactions that are involved in the pathogenesis of neurodegenerative disorders, including Alzheimer's disease (AD). Thus, the present invention is directed to complexes of these proteins and/or their fragments, antibodies to the complexes, diagnosis of neurodegenerative disorders (including diagnosis of a predisposition to and diagnosis of the existence of the disorder), drug screening for agents which modulate the interaction of proteins described herein, and identification of additional proteins in the pathway common to the proteins described herein.

The publications and other materials used herein to illuminate the background of the invention, and in particular, cases to provide additional details respecting the practice, are incorporated herein by reference, and for convenience, are referenced by author and date in the following text and respectively grouped in the appended List of References.

Alzheimer's Disease (AD) is a neurodegenerative disease characterized by a progressive decline of cognitive functions, including loss or declarative and procedural memory, decreased learning ability, reduced attention span, and severe impairment in thinking ability, judgment, and decision making. Mood disorders and depression are also often observed in AD patients. It is estimated that AD affects about 4 million people in the USA and 20 million people world wide. Because AD is an age-related disorder (with an average onset at 65 years), the incidence of the disease in industrialized countries is expected to rise dramatically as the population of these countries is aging.

AD is characterized by the following neuropathological features:

a massive loss of neurons and synapses in the brain regions involved in higher cognitive functions (association cortex, hippocampus, amygdala). Cholinergic neurons are particularly affected.

neuritic (senile) plaques that are composed of a core of amyloid material surrounded by a halo of dystrophic neurites, reactive type I astrocytes, and numerous microglial cells (Selkoe, 1994b; Selkoe, 1994a; Dickson, 1997; Hardy, Gwinn-Hardy, 1998; Selkoe, 1996b). The major component of the core is a peptide of 39 to 42 amino acids called the amyloid β protein, or Aβ. Although the Aβ protein is produced by the intracellular processing of its precursor, APP, the amyloid deposits forming the core of the plaques are extracellular. Studies have shown that the longer form of Aβ (Aβ42) is much more amyloidogenic than the shorter forms (Aβ340 or Aβ39).

neurofibrillary tangles that are composed of paired-helical filaments (PHF) (Ray et al.1998; Brion, 1998). Biochemical analyses revealed that the main component of PHF is a hyper-phosphorylated form of the microtubule-associated protein τ. These tangles are intracellular structures, found in the cell body of dying neurons, as well as some dystrophic neurites in the halo surrounding neuritic plaques.

Both plaques and tangles are found in the same brain regions affected by neuronal and synaptic loss.

Although the neuronal and synaptic loss is universally recognized as the primary cause of the decline of cognitive functions, the cellular, biochemical, and molecular events responsible for this neuronal and synaptic loss are subject to fierce controversy. The number of tangles shows a better correlation than the amyloid load with the cognitive decline(Albert, 1996). On the other hand, a number of studies showed that amyloid can be directly toxic to neurons, resulting in behavioral impairment(Ma et al. 1996). It has also been shown that the toxicity of some compounds (amyloid or tangles) could be aggravated by activation of the complement cascade, suggesting the possible involvement of inflammatory process in the neuronal death.

Genetic and molecular studies of some familial forms of AD (FAD) have recently provided evidence that boosted the amyloid hypothesis (Ii, 1995; Price et al.1995; Hardy, 1997; Selkoe, 1996a). The assumption is that since the deposition of Aβ in the core of senile plaques is observed in all Alzheimer cases, if Aβ is the primary cause of AD, then mutations that are linked to FAD should induce changes that, in a way or another, foster Aβ deposition. There are 3 FAD genes known so far (Hardy, Gwinn-Hardy, 1998; Ray et al.1998), and the activity of all of them results in increased Aβ deposition, a very compelling argument in favor of the amyloid hypothesis.

The first of the 3 FAD genes codes for the Aβ precursor, APP (Selkoe, 1996a). Mutations in the APP gene are very rare, but all of them cause AD with 100% penetrance and result in elevated production of either total Aβ or Aβ42, both in vitro (transfected cells) and in vivo (transgenic animals). The other two FAD genes code for presenilin 1 and 2 (PS1, PS2) (Hardy, 1997). The presenilins contain 8 transmembrane domains and several lines of evidence suggest that they are involved in intracellular protein trafficking, although their exact function is still unknown. Mutations in the presenilin genes are more common than in the APP genes, and all of them also cause FAD with 100% penetrance. In addition, in vitro and in vivo studies have demonstrated that PS1 and PS2 mutations shift APP metabolism, resulting in elevated Aβ42 production. For a recent review on the genetics of AD, see (Lippa, 1999).

In spite of these compelling genetic data, it is still unclear whether Aβ generation and amyloid deposition are the primary cause of neuronal death and synaptic loss observed in AD. Moreover, the biochemical events leading to Aβ production, the relationship between APP and the presenilins, and between amyloid and neurofibrillary tangles are poorly understood. Thus, the picture of interactions between the major Alzheimer proteins is very incomplete, and it is clear that a large number of novel proteins are yet to be discovered. To this end, we have initiated a systematic study looking at proteins interacting with various domains of the major Alzheimer proteins (see below). The results from these experiments provide a more complete understanding of the protein-protein interactions involved in AD pathogenesis, and thus will greatly help in the identification of a drug target. Because AD is a neurodegenerative disease, it is also expected that this project will identify novel proteins involved in neuronal survival, neurite outgrowth, and maintenance of synaptic structures, thus opening opportunities into potentially any pathological condition in which the integrity of neurons and synapses is threatened.

Thus, the picture of interactions between the major AD proteins is very incomplete, and it is clear that a number of novel proteins are yet to be discovered. Although a number of molecules have been identified as possibly involved in the disease progression, no particular protein (or set of proteins) has been identified as primarily responsible for the loss of neurons and synapses. More importantly, none of the various components identified so far in the cascade of events leading to AD is a confirmed drug target.

There continues to be a need in the art for the discovery of additional proteins interacting with various domains of the major Alzheimer proteins, including APP and the presenilins. There continues to be a need in the art also to identify the protein-protein interactions that are involved in AD pathogenesis, and to thus identify drug targets.

### SUMMARY OF THE INVENTION

The present invention relates to the discovery of protein-protein interactions that are involved in the pathogenesis of neurodegenerative disorders, including AD, and to the use of this discovery. The identification of the AD interacting proteins described herein provide new targets for the identification of useful pharmaceuticals, new targets for diagnostic tools in the identification of individuals at risk, sequences for production of transformed cell lines, cellular models and animal models, and new bases for therapeutic intervention in neurodegenerative disorders, including AD.

Thus, one aspect of the present invention are protein complexes. The protein complexes are a complex of (a) two interacting proteins, (b) a first interacting protein and a fragment of a second interacting protein, (c) a fragment of a first interacting protein and a second interacting protein, or (d) a fragment of a first interacting protein and a fragment of a second interacting protein. The fragments of the interacting proteins include those parts of the proteins, which interact to form a complex. This aspect of the invention includes the detection of protein interactions and the production of proteins by recombinant techniques. The latter embodiment also includes cloned sequences, vectors, transfected or transformed host cells and transgenic animals.

A second aspect of the present invention is an antibody that is immunoreactive with the above complex. The antibody may be a polyclonal antibody or a monoclonal antibody. While the antibody is immunoreactive with the complex, it is not immunoreactive with the component parts of the complex. That is, the antibody is not immunoreactive with a first interactive protein, a fragment of a first interacting protein, a second interacting protein or a fragment of a second interacting protein. Such antibodies can be used to detect the presence or absence of the protein complexes.

A third aspect of the present invention is a method for diagnosing a predisposition for neurodegenerative disorders in a human or other animal. The diagnosis of a neurodegenerative disorder includes a diagnosis of a predisposition to a neurodegenerative disorder and a diagnosis for the existence of a neurodegenerative disorder. In a preferred embodiment, the diagnosis is for AD. In accordance with this method, the ability of a first interacting protein or fragment thereof to form a complex with a second interacting protein or a fragment thereof is assayed, or the genes encoding interacting proteins are screened for mutations in interacting portions of the protein molecules. The inability of a first interacting protein or fragment thereof to form a complex, or the presence of mutations in a gene within the interacting domain, is indicative of a predisposition to, or existence of a neurodegenerative disorder, such as AD. In accordance with one embodiment of the invention, the ability to form a complex is assayed in a two-hybrid assay. In a first aspect of this embodiment, the ability to form a complex is assayed by a yeast two-hybrid assay. In a second aspect, the ability to form a complex is assayed by a mammalian two-hybrid assay. In a second embodiment, the ability to form a complex is assayed by measuring in vitro a complex formed by combining said first protein and said second protein. In one aspect the proteins are isolated from a human or other animal. In a third embodiment, the ability to form a complex is assayed by measuring the binding of an antibody, which is specific for the complex. In a fourth embodiment, the ability to form a complex is assayed by measuring the binding of an antibody that is specific for the complex with a tissue extract from a human or other animal. In a fifth embodiment, coding sequences of the interacting proteins described herein are screened for mutations.

A fourth aspect of the present invention is a method for screening for drug candidates which are capable of modulating the interaction of a first interacting protein and a second interacting protein. In this method, the amount of the complex formed in the presence of a drug is compared with the amount of the complex formed in the absence of the drug. If the amount of complex formed in the presence of the drug is greater than or less than the amount of complex formed in the absence of the drug, the drug is a candidate for modulating the interaction of the first and second interacting proteins. The drug promotes the interaction if the complex formed in the presence of the drug is greater and inhibits (or disrupts) the interaction if the complex formed in the presence of the drug is less. The drug may affect the interaction directly, i.e., by modulating the binding of the two proteins, or indirectly, e.g., by modulating the expression of one or both of the proteins.

A fifth aspect of the present invention is a model for neurodegenerative disorders, including AD. The model may be a cellular model or an animal model, as further described herein. In accordance with one embodiment of the invention, an animal model is prepared by creating transgenic or “knock-out” animals. The knock-out may be a total knock-out, i.e., the desired gene is deleted, or a conditional knock-out, i.e., the gene is active until it is knocked out at a determined time. In a second embodiment, a cell line is derived from such animals for use as a model. In a third embodiment, an animal model is prepared in which the biological activity of a protein complex of the present invention has been altered. In one aspect, the biological activity is altered by disrupting the formation of the protein complex, such as by the binding of an antibody or small molecule to one of the proteins which prevents the formation of the protein complex. In a second aspect, the biological activity of a protein complex is altered by disrupting the action of the complex, such as y the binding of an antibody or small molecule to the protein complex which interferes with the action of the protein complex as described herein. In a fourth embodiment, a cell model is prepared by altering the genome of the cells in a cell line. In one aspect, the genome of the cells is modified to produce at least one protein complex described herein. In a second aspect, the genome of the cells is modified to eliminate at least one protein of the protein complexes described herein.

A sixth aspect of the present invention are nucleic acids coding for novel proteins discovered in accordance with the present invention.

A seventh aspect of the present invention is a method for screening for drug candidates useful for treating a physiological disorder. In this embodiment, drugs are screened on the basis of the association of a protein with a particular physiological disorder. This association is established in accordance with the present invention by identifying a relationship of the protein with a particular physiological disorder. The drugs are screened by comparing the activity of the protein in the presence and absence of the drug. If a difference in activity is found, then the drug is a drug candidate for the physiological disorder. The activity of the protein can be assayed in vitro or in vivo using conventional techniques, including transgenic animals and cell lines of the present invention.

### DETAILED DESCRIPTION OF THE INVENTION

The present invention is the discovery of novel interactions between PS1, APP or other protein involved in AD and other proteins. The genes coding for these proteins have been cloned previously, but their potential involvement in AD was unknown. These proteins play a major role in AD and neurodegeneration, based in part on the discovery of their interactions and on their known biological functions. These proteins were identified using the yeast two-hybrid method and searching a human total brain library, as more fully described below.

Although the senile plaque density and amyloid load do not correlate with cognitive decline, the genetic data strongly support a causal involvement of amyloid production in AD pathogenesis(Neve et al. 1990; Selkoe, 1994b; Octave, 1995; Roch et al. 1993; Saitoh, Roch, 1995; Selkoe, 1994c; Selkoe, 1996a). The 3 genes identified so far that contain mutations known to cause AD are APP, PS1 and PS2. Because the number of AD mutations found in PS1 (over 50) is much larger than the number of AD mutations found in PS2 (only 2), most of the studies looking at the involvement of the presenilins in AD have focused on PS1 rather than PS2. As for APP, although the number of AD mutations in the APP gene is small (5), the mere fact the APP is the biochemical precursor of Aβ put it in the heart of countless studies world wide. Thus, it is no surprise that the APP and PS1 gene products are always found as the major components of the description of events leading to neuronal death.

APP refers to a group of transmembrane proteins translated from alternatively spliced mRNAs. The smallest isoform contains 695 amino acids and is expressed almost exclusively in the brain, where it is the major APP isoform. The other major isoforms, of 714, 751, and 770 residues, contain either one or both domains of 19 and 51 residues with homology to the OX-2 antigen and Kunitz type protease inhibitors, respectively. The metabolism of APP is complex, following several different pathways. APP can be secreted from cells such as PC12, fibroblasts, and neurons. The secretion event includes a cleavage step of the precursor, releasing a large N-terminal portion of APP, sAPP, into the medium. The majority of cleavage is at the α-secretase site and occurs within the Aβ domain between amino acids β16 and β17, and releases sAPPα extracellularly. Thus, the processing of APP through the α-secretory pathway precludes the formation of intact Aβprotein. APP can also follow a pathway that leads to the secretion of Aβ protein, as well as sAPPβ, which is 15 amino acids shorter than sAPPα. Clearly, this pathway is potentially amyloidogenic. However, the secretion of Aβ protein is not the result of an aberrant processing of APP because it occurs in cultured cells under normal physiological conditions, and secreted Aβ protein has been detected in biological fluids from normal individuals. The regulation of these two pathways involves both PKC-dependent and PKC-independent phosphorylation reactions and is also altered by some of the mutations within the APP molecule that cause AD in some Swedish families (see below).

Recently, the enzyme that cleaves APP at the β site (D597 of APP695) has been identified and its cDNA cloned (Vassar et al.1999; Hussain et al.1999). This enzyme, called BACE or Asp-2, is a transmembrane protein of 501 residues which belongs to the Aspartyl Protease family. It is unclear whether APP is the natural physiological substrate of BACE. Cleavage of APP at the a site results in the secretion of sAPPα and recycling of an 83-residue non-amyloidogenic transmembrane C-terminal fragment, C83. Cleavage of APP at the β site results in the secretion of sAPPβ and recycling of an 99-residue potentially amyloidogenic transmembrane C-terminal fragment, C99. After cleavage of the precursor at the α or β site, C83 and C99 can be further cleaved at the so called γ site (APP636 to APP638), thus releasing the p3 fragment or the Aβ peptide, respectively.

Recent studies suggest that PS1 and PS2 are capable of cleaving APP at the γ site (Wolfe et al.1999b; De Strooper et al.1999; Wolfe et al.1999a; Leimer et al.1999; Annaert et al.1999; Haass, De Strooper, 1999). However, other results argue in favor of an indirect involvement of the presenilins in APP cleavage, rather than a direct APP cleavage. The double mutation located just upstream of the β-cleavage site (known as the “Swedish” mutation) was shown to shift the metabolism of APP from the α-secretase toward the β-secretase pathway, thus increasing the production of total Aβ. On the other hand, the Val717 mutations, located just after the γ cleavage site do not alter the ratio of α vs β cleavage, but increase the ratio of Aβ42 vs total Aβ, thus making more of the highly amyloidogenic form. Therefore, both types of mutations alter the metabolism of APP in a way that results in elevated levels of Aβ42, thus fostering amyloid formation. For reviews on APP processing and its involvement in AD, see (Ashall, Goate, 1994; Selkoe, 1994b; Hardy, 1997; Selkoe, 1994c; Roch, Puttfarcken, 1996; Storey, Cappai, 1999; Haass, De Strooper, 1999; Wolfe et al.1999a; Selkoe, 1999).

There is contradicting evidence as to the cellular location where APP is cleaved by the secretases (Price et al.1995; Beyreuther et al.1996; Leblanc et al.1996; Caputi et al.1997; Selkoe, 1997). Some investigators suggested that APP is cleaved in the trans-Gogi network (TGN) or in secretory vesicles en route to the plasma membrane, while others presented evidence that intact APP reaches the plasma membrane and is cleaved only after it is expressed at the cell surface. Different cell types and expression systems could explain those discrepancies. However, it is now well established that either full-length APP or its C-terminal fragment are recycled into the endosomal-lysosomal compartment. The C-terminal fragments that contain the complete Aβ domain are transported further back to the TGN and endoplasmic reticulum, where Aβ40 and Aβ42 are produced, respectively. The free Aβ fragments are then re-routed again toward the cell surface through secretory vesicles, and ultimately secreted into the extracellular milieu, where the Aβ42 will seed the aggregation into amyloid material. Clearly, proteins that interact with the cytoplasmic tail of APP could play a major role in its intracellular traffic, thus its metabolism. The cytoplasmic domain of APP was shown to interact with intracellular proteins Fe65, Fe65L, X11, and X11L (McLoughlin, Miller, 1996; Blanco et al.1998; Russo et al.1998; Trommsdorff et al.1998). These proteins have been localized in both the cytosol and the nucleus (Zambrano et al.1998) and are thought to play a role in transcription regulation. In fact, Fe65 is known to interact with know transcription factors Mena and LSF (Zambrano et al.1998; Ermekova et al.1997). There is also ample evidence that Fe65 and LSF influence the intracellular trafficking of APP, and thus indirectly control APP metabolism (Russo et al.1998; Sabo et al.1999), a central event in AD pathogenesis.

The mechanism of Aβ toxicity is also highly controversial (Iversen et al.1995; Manelli, Puttfarcken, 1995; Gillardon et al. 1996; Behl et al. 1992; Weiss et al. 1994; Octave, 1995; Furukawa et al.1996b; Schubert, 1997). Some studies indicate that Aβ must be in the aggregated amyloid form to be toxic. Other investigators showed that soluble Aβ is toxic and suggested that aggregation of soluble Aβ into amyloid fibrils is a defense mechanism aiming at sequestering soluble Aβ. While most studies found that Aβ is toxic to cells from the outside, a few investigators also found that Aβ can kill cells from the inside, before it is secreted. Whatever the exact mechanism is, a consensus is now emerging, indicating that Aβ disrupts calcium homeostasis and triggers the generation of free radicals and lipid peroxidation (Weiss et al. 1994; Abe, Kimura, 1996; Mark et al. 1997; Kruman et al.1997). Consistent with this idea, antioxidants (such as vitamin E) and neurotrophic factors that attenuate calcium influx (such as sAPP) protect neurons from Aβ mediated toxicity (Behl et al.1992; Weiss et al.1994).

After cleavage by the α- or β-secretase, the N-terminal portion of APP is secreted into the extracellular milieu where it shows a wide variety of functions. The most relevant to AD are the neurotrophic and neuroprotective activities. A number of in vitro studies have shown that sAPP stimulates cell growth (Ninomiya et al.1993; Roch et al.1992; Saitoh et al.1989; Pietrzik et al.1998), neurite extension (Milward et al.1992; Ninomiya et al.1994; Araki et al.1991; Jin et al.1994; Yamamoto et al.1994; Small et al.1994; Li et al.1997), neuronal survival (Mattson et al.1995; Yamamoto et al.1994; Furukawa et al.1996b; Barger et al.1995), and protects neurons from various toxic insults (including glucose and/or oxygen deprivation, gp120, glutamate, Aβ) (Mattson et al.1993a; Mattson et al.1993b; Barger, Mattson, 1996; Guo et al.1998). The biochemical and cellular events underlying those in vitro activities have not been elucidated yet, however it appears that sAPP function is probably carried out by receptor mediated mechanisms and activation of a signal transduction cascade. Binding sites for sAPP were found on the surface of neuroblastoma cells, and the binding affinity was in the same range of optimal concentration (10 nM) for neurite outgrowth (Ninomiya et al.1994; Jin et al.1994).

Depending on the target cells and the experimental paradigm, sAPP was found to elicit various cellular responses that include activation of potassium channels (Furukawa et al. 1996a), activation of a membrane associated guanylate cyclase (Barger, Mattson, 1995), induction of NF-kappa B dependent transcription (Barger, Mattson, 1996), increase in phosphatidyl inositol turnover (Jin et al.1994), and changes in the phosphotyrosine balance (Wallace et al.1997b; Wallace et al.1997a; Saitoh et al.1995; Mook-Jung, Saitoh, 1997). Specifically, it was found that sAPP neurite extension activity on neuroblastoma was stimulated by genistein, a tyrosine kinase inhibitor, while orthovanadate, a phosphotyrosine phosphatase inhibitor, abolished sAPP effects (Saitoh et al. 1995). This suggests that tyrosine dephosphorylation is involved in sAPP action. On the other hand, in a different experimental paradigm, sAPP was shown to activate tyrosine phosphorylation (Wallace et al. 1997b; Wallace et al. 1997a; Mook-Jung, Saitoh, 1997), which could be the result of either inhibition of a tyrosine phosphatase, or activation of a tyrosine kinase. In any event, it is clear that sAPP modulates the balance of intracellular phosphotyrosine content. These in vitro activities are reflected in vivo by a stabilization of synaptic structures in the brain (Roch et al.1994). In addition, sAPP protected brain neurons against various injuries (Mucke et al.1995; Masliah et al. 1997) and provided neurological protection against ischemia in brain and spinal cord (Smith-Swintosky et al.1994; Bowes et al.1994; Komori et al.1997). Most importantly, these protective and trophic activities at the cellular level are reflected at the behavioral level by memory and cognitive enhancement. Specifically, sAPP was shown to increase memory retention in rats (Roch et al. 1994; Gschwind et al.1996; Huber et al.1997) and mice (Meziane et al.1998), and conversely, compromising the function of sAPP resulted in memory and learning impairment (Huber et al. 1993; Doyle et al.1990). The site of sAPP that is responsible for the trophic activity was mapped to a domain of 17 amino acids, from Ala319 to Met332. This peptide was shown to stimulate cell growth, to bind to neuroblastoma cells and trigger neurite extension, to enhance neuronal survival, synaptic stability, and memory retention (Roch et al. 1994; Ninomiya et al. 1994; Jin et al. 1994; Ninomiya et al. 1993; Yamamoto et al. 1994). Furthermore, this sAPP peptide was shown to elicit the same cellular responses as sAPP itself, namely the increase in phosphatidyl inositol turnover (Jin et al. 1994) and changes in tyrosine phosphorylation (Saitoh et al. 1995; Mook-Jung, Saitoh, 1997). In brief, there is now mounting evidence for a neurotrophic and neuroprotective function of sAPP, which is reflected by increased learning and memory performance.

A few years ago, two new Alzheimer genes were discovered, coding for PS1 and PS2 (Hardy, 1997; Hardy, Gwinn-Hardy, 1998; Ray et al.1998). These two proteins share 67% identity and although a number of studies report a topological structure with 6 to 9 transmembrane domains, a consensus is now emerging for a structure with 8 transmembrane domains(Doan et al.1996; Lehmann et al.1997; Hardy, 1997). Although their exact function is not known, they appear to be involved in intracellular protein trafficking. Thus, presenilins are potentially implicated in APP metabolism. This hypothesis is supported by numerous in vitro and in vivo studies showing that the AD mutations in PS1 and PS2 alter APP metabolism resulting in elevated production of Aβ42, although the total Aβ was not changed (Duff et al. 1996; Lemere et al. 1996; Borchelt et al. 1996; Tomita et al.1997; Ishii et al.1997; Oyama et al.1998; Hutton, Hardy, 1997; Cruts, Van Broeckhoven, 1998; Kim, Tanzi, 1997; Hardy, 1997; Citron et al.1998).

The possibility that PS1 and PS2 function as APP cleaving enzymes was recently raised by a number of investigators (De Strooper et al.1999; Wolfe et al.1999a; Sinha, Lieberburg, 1999; Annaert et al.1999; Haass, De Strooper, 1999), although it is most widely accepted that the presenilins actually control the activity of γ-secretase(s) rather than cleave APP directly. Still, the mere fact that AD mutations in proteins other than APP itself also result in increased production of Aβ42 is a compelling argument in favor of the amyloid hypothesis. Additionally, mutations in PS-1 and PS-2 have been shown to be neurotoxic through an apoptotic mechanism that is independent of amyloid production, notably the generation of superoxide and disruption of calcium homeostasis (Vito et al.1996; Wolozin et al.1996; Zhang et al.1998; Renbaum, Levy-Lahad, 1998; Guo et al.1998; Mattson, 1997a; Guo et al.1999a; Guo et al.1999b; Guo et al.1996). Recent studies have shown that the presenilins bind to several proteins of the Armadillo family, including β-catenin, δ-catenin, and p0071(Yu et al.1998; Murayama et al.1998; Zhou et al.1997; Levesque et al.1999; Tanahashi, Tabira, 1999; Stahl et al. 1999). The biological significance of these interactions is not clear, although recent studies suggest that FAD presenilin mutations disrupt the normal interaction pattern of the Armadillo proteins, and lead neuronal apoptosis (Zhang et al. 1998; Tesco et al. 1998). For example, the presence of PS-1 and β-catenin in the same complex could influence the ultimate fate of β-catenin and its involvement with axin, GSK3-β, and PP2A in the wingless signaling pathway (Nakamura et al.1998; Kosik, 1999; Dierick, Bejsovec, 1999). Conceivably, FAD associated mutations in PS1 could disrupt the PS1-β-catenin complex, resulting in aberrant β-catenin mediated signalling and eventual neuronal death.

In brief, there is now growing evidence that APP metabolism and Aβ generation are central events to AD pathogenesis, and that mutations in the presenilins can induce neuronal apoptosis as well as stimulate amyloid deposition. However, many obscure points remain. Although a candidate β-secretase enzyme has been identified, its normal physiological substrate is not known. Even less is known about the α- and γ-secretases (with the reservation concerning the potential role of PS1 and PS2 as γ-secretase, mentioned above). A direct biochemical link between the presenilins and APP processing has not been firmly established. The proteins that mediate the neurotrophic and neuroprotective effects of sAPP are unknown. This last point is of utmost importance because an alteration of APP metabolism could result in both the generation of a toxic product (Aβ) and the impairment of SAPP trophic activity (Saitoh et al.1994; Roch et al.1993; Saitoh, Roch, 1995). In this respect, it is interesting that one APP mutation associated with Alzheimer's results in a defective neurite extension activity of sAPP (Li et al. 1997). Moreover, the balance of phosphorylation cascades is deeply altered in Alzheimer brains (Saitoh, Roch, 1995; Jin, Saitoh, 1995; Mook-Jung, Saitoh, 1997; Saitoh et al.1991; Shapiro et al.1991). Because hyperphosphorylation of the microtubule-assiciated protein τ is necessary for the formation of paired helical filaments and tangles, a disruption of the phosphorylation cascade could be the link between the amyloid and the τ pathways.

Proteins that interact with sAPP are expected to be involved in its biological function, including neuron survival, synaptic formation and stability, learning and memory. Thus, it is expected that some of these will become promising targets for drugs designed to tackle AD and a number of other neurodegenerative conditions. Because sAPP showed obvious protective effects in ischemia models (Smith-Swintosky et al.1994; Bowes et al.1994; Mattson, 1997b; Komori et al. 1997), it is reasonable to assume that drugs that mimic sAPP function could be used to alleviate the effects of stroke (Mattson, 1997b). Likewise, the discovery of new proteins that interacts with the presenilins, δ-catenin, Fe65, or axin could establish previously unknown biochemical pathways, and identify drug targets that could influence APP metabolism, presenilin functions, neuronal survival, and synaptic maintenance. As mentioned above, cholinergic neurons are particularly affected and levels of acetylcholine are markedly reduced in AD brains compared to controls. To date, the only Alzheimer drugs available are inhibitors of acetylcholine esterase (AChE). This enzyme has also been found to be associated with neuritic plaques (Inestrosa, Alarcon, 1998) and to interact with APP (Alvarez et al. 1998). Thus, proteins that interact with AChE also represent important opportunities for drug discovery in Alzheimer's disease.

According to the present invention, new protein-protein interactions have been discovered. The discovery of these interactions has identified several protein complexes for each protein-protein interaction. The protein complexes for these interactions are set forth below in Tables 1-37, which also identify the new protein-protein interactions of the present invention. The involvement of the protein-protein interactions in neurodegenerative disease is described below with reference to individual or grouped interactions.

TABLE 1
Protein-Protein Interactions of PS1-FKBP25

Presinilin 1 (PS1) and Rapamycin-binding protein 25 (FKBP25)
A fragment of PS1 and FKBP25
PS1 and a fragment of FKBP25
A fragment of PS1 and a fragment of FKBP25

TABLE 2
Protein Complexes of FKBP25-CIB Interaction

Rapamycin-binding protein 25 (FKBP25) and CIB
A fragment of FKBP25 and CIB
FKBP25 and a fragment of CIB
A fragment of FKBP25 and a fragment of CIB

Immunosuppressant drugs such as FK506, rapamycin, and cyclosporine A act by inhibiting T cell proliferation and bind to a group of proteins collectively called immunophilins. Although most of the studies on immunophilins have focused on lymphocytes, the recent finding that immunophilins are much more abundant in the nervous system than the immune system has opened promising new therapeutic avenues(Snyder et al.1998; Steiner et al.1997a; Steiner et al.1997b). In the immune system, cyclosporine A (CsA) and FK506 inhibit the synthesis and secretion of interleukin-2 (IL-2), an early step in the response of T cells to antigen. Rapamycin, on the other hand, blocks the IL-2-induced clonal proliferation of activated T cells by inhibiting signaling through the IL-2 receptor. These findings suggested that CsA and FK506 may act through similar molecular mechanisms, while rapamycin act through a different mechanism(Snyder et al. 1998). It was found that CsA binds to an 18 kDa protein called cyclophilin, and FK506 binds to a 12 kDa protein called FKBP12. Both cyclophilin and FKBP12 show peptide-propyl isomerase (rotamase) activity(Snyder et al.1998). Although the immunophilin ligands inhibit the rotamase activity, several of these ligands lack immunosuppressant activity. This indicated that the rotamase activity is not linked to the immunosuppressant effect. The drug-immunophilin complex was suggested to acquire a gain of function and bind to another protein that neither the drug or the immunophilin alone would interact with. The first drug-immunophilin target was identified as calcineurin, a Ca²⁺-calmodulin activated phosphatase. Calcineurin was found to bind both CsA-cyclophilin A complexes and FK506-FKBP12 complexes(Cameron et al.1995). One of the calcineurin substrates is the phosphorylated form of the transcription nuclear factor of activated t-cells (NF-AT) which is known to activate transcription of many genes in T-cells, including IL-2 and its receptor. Only the non-phosphorylated form of NF-AT can enter the nucleus. Binding of drug-immunophilin complexes to calcineurin inhibits its activity, leading to elevated phosphorylation levels of NF-AT and in reduced transcription of IL-2 and its receptor (as NF-AT is then not able to enter the nucleus). As for rapamycin, it was shown also to bind FKBP12 with very high affinity. The complex does not bind to calcineurin but to a group of proteins called rapamycin and FKBP12 target 1 (RAFT1), FKBP and rapamycin associated protein (FRAP), and mammalian target of rapamycin (TOR) (Freeman, Livi, 1996; Lorenz, Heitman, 1995). RAFT1 is known to phosphorylate the protein translation regulator 4E-BP1(Snyder et al.1998).

In the nervous system, immunophilin concentrations are 50 fold higher than in the immune system(Snyder et al. 1998). Both cyclophilin and FKBP-12 are almost exclusively neuronal in the brain, with striking regional variations that closely resemble those of calcineurin. Highest levels are found in the granular cells of the cerebellar folia, in the hippocampus, in the striatum, and in the substantia nigra. Two major brain substrates of calcineurin are GAP-43 (mediating neurite outgrowth) and neuronal nitric oxide synthase (nNOS). Nitric oxide is a mediator of glutamate induced toxicity through NMDA receptors, as nNOS inhibitors and nNOS gene knockout can block this toxic effect. nNOS activity is inhibited when the enzyme is phosphorylated. Therefore, nNOS is expected to be activated by calcineurin, and blocked by calcineurin inhibitors(Snyder et al. 1998; Steiner et al.1997a; Steiner et al.1997b). Indeed, by inhibiting calcineurin, FK506 was shown to increase the levels of phosphorylated nNOS, thus reducing its catalytic activity, and providing neuroprotection against glutamate. As expected, rapamycin blocked the effect of FK506 (since it binds to FKBP12 but the FK506-FKBP12 complex does not bind to calcineurin). Another effect of FK506 in brain is the modulation of neurotransmitter release. As nitric oxide is also required for neurotransmitter release from PC12 cells and brain synaptosomes stimulated by NMDA, FK506 inhibits neurotransmitter release in these systems, and these effects are blocked by rapamycin. By contrast, neurotransmitter release is stimulated by FK506 in synaptosomes depolarized by K+channel blockers. This effect is mediated by synapsin I, a synaptic vesicle associated protein, and dynamin I, a GTPase involved in the recycling of synaptic vesicles. The neurotransmitter release activity of both proteins is stimulated by phosphorylation and inhibited by dephosphorylation. Since both synapsin I and dynamin I are substrates for calcineurin, inhibition of the phosphatase activity of calcineurin by FK506 increases the phoshporylation state of synapsin I and dynamin I, thus stimulating neurotransmitter release. Another important effect of the immunophilins in the brain is the modulation of intracellular concentration of Ca²⁺(iCa²⁺). FKBP12 binds to the ryanodine receptor and to the IP3 receptor, two proteins involved in the release of Ca²+from intracellular stores. Both receptors are activated when phosphorylated by the protein kinase C (PKC). The binding of FKBP12 to these receptors attracts calcineurin in the complex, which reduces the phosphorylation level of the receptor. In the presence of FK506, the FKBP12-calcineurin complex dissociates from the IP3 receptor, which shows increased activity, resulting in elevated iCa²⁺(Snyder et al.1998; Steiner et al.1997a; Steiner et al.1997b).

In addition FK506 also has neurotrophic activities that were observed in PC12 cells and sensory ganglia at subnanomolar concentration, similar to well characterized neurotrophic factors such as the nerve growth factor (NGF), brain-derived growth factor (BDNF), and neurotrophins NT-3 and NT-4. Recently, FK506 derivatives were synthesized that bind immunophilins (FKBP12) with the same potency as the parent drug, but the drug-immunophilin complexes did not bind calcineurin and had no immunosuppressant activity. However, these new drugs (e.g. GPI1046) retained the full neurotrophic activity of FK506. Stimulation of neurite outgrowth was observed at 1 pM concentration, with a maximal effect at 1 nM. Furthermore, while the classic neurotrophic proteins (NGF, BDNF, NT-3 and NT-4) each act only in a selected repertoire of neuronal systems, immunophilin ligands (FK506 and derivatives) are active in all the systems examined. However, the neurotrophic actions of the immunophilin ligands are restricted to damaged neurons, but have no effect on normal peripheral or central neurons (while neurotrophic proteins elicit such effect). Thus, immunophilins mediate both calcineurin-dependent and calcineurin-independent neurotrophic activities(Snyder et al. 1998; Steiner et al.1997a; Steiner et al.1997b).

In a yeast 2-hybrid search using the amino-terminal cytosolic region of presenilin-1 (aa 1 to 91), we isolated a clone corresponding to the carboxy-terminal region (aa 166 to 224) of FKBP25. This protein, in the same family as FKBP12, is an immunophilin that binds FK506 and rapamycin, and has a rotamase domain in its C-terminal half(Jin et al. 1992; Galat et al. 1992; Hung, Schreiber, 1992; Wiederrecht et al.1992). It shares about 45% identity with other FKBP proteins (FKBP12,-13, and -59) in the 97 C-tenninal residues, while it's amino terminal region does not share identity or similarity with any known protein. As for other FKBP proteins, FKBP25 rotamase activity is inhibited by both FK506 and rapamycin, however rapamycin has a much greater potency (IC₅₀ is 50 nM) than FK506 (IC₅₀ is 400 nM) (Jin et al.1992; Galat et al.1992; Hung, Schreiber, 1992; Wiederrecht et al. 1992). The cellular and biochemical mechanisms elicited by FKBP25 are at present unknown. Because FKBP12-rapamycin complexes do not act through the calcineurin pathway, and because FKBP25 has a much higher affinity for rapamycin than for FK506, it is likely that FKBP25 acts predominantly through calcineurin-independent pathways, and to a lesser extend through calcineurin-dependent pathways. Indeed, FKBP25 contains a nuclear localization signal in its rotamase domain (which is absent in other FKBPs), was localized in the nucleus, and binds to casein kinase II (CKII) and nucleolin(Jin, Burakoff, 1993). CKII phosphorylates a number of cytosolic and nuclear substrates, and is an important regulator of cell growth. The phosphorylation of nucleolin is a crucial step in ribosome formation. It is possible that the phosphorylation of FKBP25 enhances its translocation to the nucleus, and in turn, the association of CKII with FKBP25 could also facilitate the nuclear translocation of the kinase, which could then phosphorylate nucleolin and other nuclear substrates. Alternatively, the rotamase activity of FKBP25 could inhibit the function of CKII and nucleolin. The high levels of FKBP25 in hippocampus (a severely affected area in AD brain) and its association with PS-1 and with CKII suggests that FKBP25 is involved in a brain function that is related to Alzheimer's disease. FKBP25 belongs to the immunophilin family, whose neurotrophic actions have been well documented, and it may play a critical role in the survival of hippocampal neurons. In this respect, its association with wild-type or mutant forms of PS-1 could alter its activity. The activity and protein levels of CKII are greatly reduced in AD brains, and this reduction closely matches the regional distribution of the pathological features. One of the target of CKII is APP, and it is known that APP phosphorylation affects its metabolism. Thus, PS-1 mutations could alter the function of FKBP25, which in turn could change the activity of CKII, and ultimately the phosphorylation state of APP, its metabolism, and the production of Aβ. Alternatively, the alteration of FKBP25 function (because of an altered interaction with FAD mutant PS-1) could destabilize calcium homeostasis and lead directly to neuronal apoptosis. Thus, the biological effects elicited by FKBP25 may be of great importance for neuron survival and their alteration may be critical in neurodegenerative processes like those observed in Alzheimer.

As a first step toward a better understanding of the cellular and biochemical events elicited by FKBP25, we performed a yeast two-hybrid search against a brain library using the full-length FKBP25 protein as a bait, and isolated a clone coding for a calcium binding protein called CIB. Further characterization using shorter FKBP25 fragments as baits showed that the 25 N-terminal residues of FKBP25 also interacts with CIB. This suggests that CIB may interact specifically with FKBP25 but no other FKBPs, as the N-terminal region of FKBP25 is not shared with other FKBPs. CIB is a 191 amino acid protein that was discovered in 1997 in a yeast two-hybrid search using the cytoplasmic domain of integrin αIIb as a bait (Naik et al. 1997). CIB contains 2 calcium binding domains (EF hands) and is 58% similar (28% identical) to calcineurin B, the 19 kDa regulatory subunit of calcineurin; and 55% similar (27% identical) to calmodulin. The authors of this study suggest that CIB might be the regulatory subunit of a new, as yet unknown, multi-subunit calcium-dependent phosphatase. Because other FKBPs are known to bind the IP3 and the ryanodine receptors, it is also possible that FKBP25, CIB and its associated phosphatase bind to and control the phosphorylation state of the IP3 or the ryanodine receptors. Thus, the PS1-FKBP25-CIB pathway could play a major role in the control of calcium release from internal stores. In support of this hypothesis, PS1 was recently shown to bind the ryanodine receptor directly (Mattson et al. 1999), and this interaction was shown to control calcium homeostasis. In addition, CIB was recently shown to interact with PS2 and PS1 (Stabler et al.1999). FAD associated mutations in PS1 and PS2 induce neuronal apoptosis through the disruption of neuronal calcium homeostasis. It is likely that these mutations disrupt the interactions of PS1 and PS2 with other proteins, like FKBP25, CIB, and the ryanodine receptor. Thus, the interaction network generated by our findings provides a direct biochemical link between the presenilins and the control of calcium homeostasis. Pharmacological agents that influence these protein-protein interactions will play a major role in the control of neuronal survival or apoptosis.

TABLE 3
Protein Complexes of PS1-rab 11 Interaction

PS1 and the carboxy-terminal region of rab-related GTP-binding
protein 11 (rab 11)
A fragment of PS1 and rab 11
PS1 and a fragment of rab 11
A fragment of PS1 and a fragment of rab 11

TABLE 4
Protein Complexes of APP-BAT3 Interaction

Amyloid precursor protein (APP) and HLA-B associated transcript
(BAT3)
A fragment of APP and BAT3
APP and a fragment of BAT3
A fragment of APP and a fragment of BAT3

TABLE 5
Protein Complexes of BAT3-δ-adaptin Interaction

HLA-B associated transcript (BAT3) and δ-adaptin
A fragment of BAT3 and δ-adaptin
BAT3 and a fragment of δ-adaptin
A fragment of BAT3 and a fragment of δ-adaptin

As described above, the intracellular traffic of APP is quite complex. After secretion of the large N-terminal fragment by the α- or β-secretase, the transmembrane C-terminal fragment (which may or may not contain the entire Aβ region) is endocytosed into clathrin-coated pits, and targeted to other intracellular compartments(Selkoe et al. 1996c; Selkoe, 1994c). Some cells have a low secretory activity and also recycle full-length APP back into the intracellular membrane network. Because the final destination of each fragment will determine its eventual fate, the intracellular trafficking of APP metabolites is a very important event leading to the production of the Aβ peptide, and its release from the cells. APP and its metabolites have been detected in almost all intracellular compartments, like the recycling endosomes (going to the Golgi and endoplasmic reticulum (ER)), and sorting endosomes (going to the lysosomes or back to the plasma membrane). While the pathways going from the plasma membrane to the Golgi and ER or to the lysosomes are responsible for Aβ production or degradation, the recycling route toward the membrane is a crucial step potentially leading to Aβ secretion(Selkoe, 1998). Thus, any protein involved in the traffic of intracellular vesicles containing APP metabolites could play a major role in the production and release of Aβ.

Small GTPases of the rab family play an essential role in the control of intracellular vesicle trafficking(Geppert, Sudhof, 1998). These proteins are expressed at high levels in the neuro-endocrine system and they represent crucial elements regulating processes like hormone secretion and neurotransmitter release(Deretic, 1997). Over 30 different rab proteins have been identified, showing a wide range of expression, from gastric wall to brain, and different distribution into distinct subcellular compartments. This suggests that different members of the rab family might confer specificity to particular intracellular pathways. However, the detailed molecular mechanisms of action of the rab proteins are not completely understood. The rab3 protein is involved in the fusion of neurotransmitter-loaded secretory vesicles with the plasma membrane, an event which involves GTP hydrolysis, GDP/GTP exchange with the protein GDI, and an elevation of Ca²⁺in the synaptic terminal(Park et al.1997; Johannes et al.1994; Ahnert-Hilger et al. 1996; Geppert, Sudhof, 1998). Several isoforms of rab3 have been described, but the specific function of each one of them is not known yet. It is nevertheless clear that rab3 is involved in neurotransmitter release. Other rab proteins such as rab4, rab5, rab11, rab17, rab18, and rab20 have all been shown to be involved in a complex endocytotic pathway(Geppert, Sudhof, 1998), and different rab proteins associate with endosomes targeted to specific subcellular compartments. A number of studies have shown that rab11 associates with recycling endosomes and other post-Golgi membranes such as the trans-Golgi network (TGN) and secretory vesicles. On the other hand, the rab5 protein is associated with sorting endosomes (en route to the lysosomes) and other early factors of the endocytotic traffic. To date, rab11 is the only GTPase known to regulate the intracellular traffic through recycling endosomes(Ullrich et al.1996).

A number of mutations in PS1 are known to cause Alzheimer Disease in some families. Both in vitro (cell transfection) and in vivo (transgenic mice) studies have shown that these mutations result in an increase of Aβ42 production and secretion(Duff et al. 1996; Hutton, Hardy, 1997; Cruts, Van Broeckhoven, 1998; Kim, Tanzi, 1997; Hardy, 1997; Selkoe, 1998), which is an evidence of an alteration of APP processing. However, the existence of a direct biochemical link between APP and PS1 is still highly controversial, and it is not clear at all how mutations in PS1 could alter APP metabolism. A recent study(Wolfe et al. 1999b) suggested that PS1 could be the γ-secretase itself, although it is equally possible that PS1 is a regulatory protein that modulates the activity of γ-secretase. In a yeast 2-hybrid search using the amino-terminal cytosolic region of presenilin-1 (aa 1 to 91), we isolated a clone corresponding to the carboxy-terminal region (aa 106 to 216) of rab11(Gromov et al.1998; Lai et al.1994; Urbe et al.1993; Sheehan et al.1996). The discovery of a direct biochemical interaction between PS1 and rab11 offers an attractive explanation of the mechanism whereby PS1 mutations cause elevated secretion of Aβ42. As described above, rab11 controls the trafficking of recycling endosomes and targets proteins to the Golgi and ER. The cytoplasmic domain of APP is known to interact with the protein Fe65, which in turn interacts with LSF(Russo et al.1998). As described herein LSF interacts with both APP and PS1. Thus, the interaction series APP→Fe65→LSF→PS1→rab11 suggests that upon endocytosis, APP can be driven to the Golgi and endoplasmic reticulum through rab11-containing recycling endosomes. It is expected that mutations in PS1 could alter its interactions with other proteins, including rab11. This in turn could change the ultimate fate of APP-containing vesicles: if the PS1-rab11 interaction is tight, the endocytic vesicles will go to the Golgi and ER compartment. On the other hand, if the PS1-rab11 interaction is lose, the vesicles will become sorting endosomes and go either back to the plasma membrane (a rare event) or to the lysosomes, where APP and its metabolites are completely degraded. This model predicts that the interaction of APP with Fe65 would promote the production of the Aβ peptide, which was confirmed recently(Sabo et al. 1999). On the other hand, driving APP away from the Golgi-ER compartment and toward lysosomes is expected to reduce Aβ production. This is indeed what was observed(Schrader-Fischer et al.1997).

Using the C-terminal cytoplasmic fragment of APP-695 as a bait (aa 639 to 695), we identified a clone encoding amino acids 603 to 1132 (C-terminal) of the BAT3 protein. Also called HLA-B associated transcript 3, BAT3 is a protein of unknown function that contains a ubiquitin-like domain in the N-terninal region (aa 17 to 77) and two proline-rich domains (aa 202 to 207 and 657 to 670) (Banerji et al.1990; Wang, Liew, 1994; Spies et al.1989b; Spies et al.1989a). Thus, the domain of BAT3 that interacts with APP contains the second proline-rich region, but not the ubiquitin-like domain. As mentioned in the Background section, APP is involved in a wide variety of functions throughout the organism. Like APP, BAT3 is expressed in all tissues examined, including brain. Thus, BAT3 might be involved in APP recycling or intracellular trafficking which, as discussed above, is a crucial event that modulates Aβ production. To find out if and how BAT3 interaction with APP could influence APP trafficking, we looked for proteins that interact with BAT3. Using the N-terminal domain of BAT3 (aa 1 to 241) as a bait in a yeast two-hybrid search, we identified a clone coding for amino acids 1062 to 1153 of 6-adaptin. This protein is the major component of the AP-3 complex (Dell'Angelica et al.1998). Transport vesicles are coated by clathrin and by associated protein complexes known as AP-1, AP-2, AP-3, and AP-4 (Hirst, Robinson, 1998). Each of these complexes contains a specific set of proteins having extensive sequence similarity with one other. The most notorious of these proteins are called adaptins. Adaptin α and γ are components of the AP-1 and AP-2 complexes, respectively, while d-adaptin is part of the AP-3 complex. A recent study (Le Borgne et al. 1998) showed that the AP-3 complex mediates the intracellular transport of transmembrane glycoproteins to lysosomes. Thus, because BAT3 interacts with the cytoplasmic domain of APP, the BAT3-δ-adaptin connection could be a key to the lysosomal targeting of APP. This is of utmost importance because targeting APP to the lysosomal compartment reduces Aβ secretion(Schrader-Fischer et al. 1997).

In summary, during endocytosis, APP can be targeted to recycling or sorting endosomes. The recycling endosomal vesicles eventually go to the Golgi and the ER, where Aβ40 and Aβ42, respectively, are made. On the other hands, sorting endosomes can either go directly back to the plasma membrane (a rare event) or to lysosomes, where APP metabolites are degraded. The rab11 GTPase (a PS1 interactor) is highly enriched in recycling endosomes vs sorting endosomes, and thus may be involved in targeting APP to cell compartments that produce Aβ. Therefore, a new model of APP trafficking emerges, in which rab11 and PS1 interact with APP (through the Fe65-LSF connection), targeting it to recycling endosomes, while the BAT3-δ-adaptin complex brings APP to sorting endosomes and lysosomes, where no Aβ is produced. Thus, APP trafficking and metabolism may be controlled by a competitive interaction with BAT3 or Fe65. In this respect, pharmacological agent that favor the BAT3-APP interaction are expected to drive APP to the lysosomes, thus reducing Aβ production.

In addition, BAT3 could also be involved in the brain-specific (neurotrophic, synaptotrophic) functions of APP. Using yeast two-hybrid system and co-immunoprecipitation, a recent study showed that the domain of BAT3 from aa 246 to 360 bind to CAP1, an adenylate cyclase associated protein(Hubberstey et al.1996). CAP1 is a 475 amino acid protein with two functionally different domains separated by a proline-rich region. Studies on yeast CAP showed that the N-terminal domain is involved in activation of adenylate cyclase while the C-terminal domain is involved in nutritional and temperature sensitivity, growth, cell morphology, and budding(Zelicof et al. 1996). In this respect, it is interesting that the random budding phenotype, observed in yeast strains that do not express CAP, could be suppressed by over expression of SNC 1, a yeast homolog of mammalian synaptobrevin, a protein involved in the fusion of synaptic vesicles with the presynaptic membrane. It is thus possible that in human, CAP1 and synaptobrevin are involved in similar aspects of synaptic formation and maintenance. As for the activity of the N-terminal fragment of CAP1, the activation of adenylate cyclase results in elevation of intracellular cAMP levels, a phenomenon that has been linked to long-term potentiation (LTP) (Sah, Bekkers, 1996; Kimura et al.1998; Storm et al.1998; Villacres et al.1998), considered as the cellular and biochemical substrate for memory(Matzel et al. 1998; Davis, Laroche, 1998). Thus, APP (a protein directly involved in AD and with well documented brain functions) interacts with BAT3, a large proline-rich protein. BAT3 in turn interacts with CAP1, another proline-rich protein containing one domain involved in the regulation of cAMP levels (thus influencing LTP and memory) and another domain that, like synaptobrevin, might participate in synaptic functions. Thus, BAT3 represents a crucial link between APP and CAP1, two proteins with brain specific functions. The BAT3-APP interaction is thus a potential point of intervention in the biochemical and cellular events leading to synaptic formation and LTP (memory), with a direct impact on Alzheimer's disease.

Considering the potential effects of BAT3 on both APP metabolism and APP neurotrophic function, as described above, drugs that would favor the BAT3-APP interaction are useful against the neurodegeneration observed in Alzheimer's patients.

TABLE 6
Protein Complexes of APP-PTPZ Interaction

Amyloid precursor protein (APP) and protein tyrosine phosphatase zeta
(PTPZ)
A fragment of APP and PTPZ
APP and a fragment of PTPZ
A fragment of APP and a fragment of PTPZ

The protein tyrosine phosphatase zeta (PTPZ, Swiss-Prot accession number: P23471; GenBank accession number: M93426) is a large type I transmembrane protein of 2314 amino acids, expressed specifically in the central nervous system (Krueger and Saito, 1992; Shintani et al., 1998). It has the typical structure of a cell surface receptor, with a signal peptide from amino acids 1 to 24 and a single transmembrane domain from amino acids 1636 to 1661. Amino acids 25 to 1635 are extracellular, while amino acids 1662 to 2314 are cytoplasmic. Two tyrosine phosphatase domains are from amino acids 1744 to 1997 and from amino acids 1998 to 2314. Interestingly, PTPZ expression is increased in response to injury (Li et al., 1998). It is also expressed at high levels by neurons and astrocytes during brain development. PTPZ belongs to a large family of phosphatases that play important roles in neuronal functions. Using a domain from amino acids 306 to 500 of APP695 as a bait in a yeast two-hybrid search, we identified a clone coding for a domain of PTPZ from amino acids 1052 to 1128. As mentioned above, the secreted form of APP695 (which includes amino acids 306 to 500) has well documented neurotrophic activities, and a large body of evidence indicates that these activities are carried out by receptor mediated mechanisms. Moreover, the balance of tyrosine phosphorylation was shown to mediate sAPP neurotrophic activity. However, no APP receptor protein has been described yet. Thus, the finding that sAPP binds an extracellular domain of PTPZ provides the first biochemical link to the cellular mechanisms that underlie sAPP activity. Because APP metabolism and function as well as phosphorylation reactions are deeply disrupted in the brain of Alzheimer's patients, and because sAPP activities at the cellular level (neurotrophic, neuroprotective) are reflected by memory enhancement at the behavioral level, it is expected that drugs that alter PTPZ activity will have a tremendous potential for the treatment of neurodegenerative disease, in particular Alzheimer's disease.

TABLE 7
Protein Complexes of APP695-KIAA0351 Interaction

Amyloid Aβ protein precursor, 695 isoform (APP695) and KIAA0351
A fragment of APP695 and KIAA0351
APP695 and a fragment of KIAA0351
A fragment of APP695 and a fragment of KIAA0351

The sequence reported in GenBank (AB002349) for KIAA0351 is 6.3 kb long and contains an ORF coding for 557 residues, with an ATG initiation codon in a reasonably good Kozak environment (A in position −3). Our interacting clone encodes aa 213 to 557, the C-terminus. Because the KIAA0351 protein is novel, nothing is known about its biological function. Amino acid sequence analysis revealed the presence of a pleckstrin homology (PH) region, between aa 431 and 480. According to the Prosite documentation (PDOC 50003), the PH domain is found in a variety of proteins involved in intracellular signaling or that are components of the cytoskeleton. For example, many proteins with GTPase activity, or GTP exchange factors contain PH domains. This feature is particularly relevant to the neurotrophic and neuroprotective functions of sAPP which could be mediated by a membrane-associated guanylate cyclase and formation of cGMP (Barger, Mattson, 1995; Barger et al.1995). In this respect, KIAA0351 could represent a GTP donor that the guanylate cyclase could use as a substrate to form cGMP, upon activation by sAPP. KIAA0351 share 48% similarity with GNRP, a guanine nucleotide releasing protein. A PH domain was also found in the Insulin Receptor Substrate 1 (IRS-1), which is important in the light of a study that showed that sAPP neurotrophic activity is mediated by phosphorylation of IRS-1 (Wallace et al.1997). In brief, we have identified an interaction between the neurotrophic region of sAPP and a protein of unknown function, KIAA0351. The presence of a PH domain in KIAA0351 suggests that this protein can mediate the neurotrophic effect of sAPP.

TABLE 8
Protein Complexes of APP695-Prostaglandin D Synthase Interaction

Amyloid Aβ protein precursor, 695 isoform (APP695) and
Prostaglandin D synthase
A fragment of APP695 and Prostaglandin D synthase
APP695 and a fragment of Prostaglandin D synthase
A fragment of APP695 and a fragment of Prostaglandin D synthase

The interaction of APP695 and prostaglandin D synthase is important in the light of the well documented inflammatory component of the Alzheimer pathology (Yamada et al. 1996; Kalaria et al.1996b; Kalaria et al.1996a; Dickson, 1997; Cummings et al.1998). The intricate cross-talks between the amyloid pathway and inflammation pathway make the situation complex. Beside the generation of free radicals, lipid peroxidation, and disruption of calcium homeostasis (Manelli, Puttfarcken, 1995; Weiss et al.1994; Mark et al.1997; Mark et al.1995; Mattson, 1997a), there is evidence that Aβ toxicity can be mediated in part by some inflammatory factors (Fagarasan, Aisen, 1996; McRae et al.1997) including components of the complement cascade (Pasinetti, 1996). Furthermore, cyclo-oxygenase 1 and 2 (COX1 and COX2) activities are elevated in Alzheimer brains and prostaglandins are known neurotoxins (Prasad et al. 1998; Pasinetti, Aisen, 1998; Lee et al. 1999; Kitamura et al. 1999). Reciprocally, factors released by activated microglial cells appear to accelerate the transition of diffuse plaques into mature neuritic plaques observed in AD brains (Sheng et al. 1997). The secreted form of APP (sAPP) has well documented survival, neurotrophic, and neuroprotective activities (Roch et al.1993; Saitoh, Roch, 1995; Roch, Puttfarcken, 1996; Goodman, Mattson, 1994; Mattson et al.1993; Mattson, 1997c). These effects at the cellular levels are reflected by memory enhancement at the behavioral levels (Roch et al. 1994; Meziane et al. 1998; Huber et al.1997; Roch, Puttfarcken, 1996; Huber et al.1993). The domain involved in these activities was localized between the residues Ala319 and Met335 of APP695 (Roch et al.1993; Saitoh, Roch, 1995; Roch, Puttfarcken, 1996), which is part of the bait that we used to identify prostaglandin D synthase as an interactor. The sAPP interaction with prostaglandin D synthase is believed to control prostaglandin D synthesis. Because prostaglandins can be neurotoxic, drugs that modulate the activity of prostaglandin D synthase or its interaction with APP could be used to reduce the levels of prostaglandin D in the brain, and alleviate the prostaglandin-mediated neurotoxicity. Additionally, the preferential localization of prostaglandin D in brain makes it an attractive drug target.

TABLE 9
Protein Complexes of AChE-Calpain small subunit Interaction

Acetylcholine esterase (AChE) and Calpain small (regulatory) subunit
A fragment of AChE and Calpain small (regulatory) subunit
AChE and a fragment of Calpain small (regulatory) subunit
A fragment of AChE and a fragment of Calpain small (regulatory) subunit

The calcium-activated neutral proteinase (CANP) calpain, an enzyme involved in intracellular signaling, is a heterodimer of a large (80 kDa) catalytic and small (30 kDa) regulatory subunits (Suzuki et al.1995). The catalytic subunit exists in 2 variants, μ- and m-, activated by micromolar and millimolar calcium concentrations, respectively. The physiological function of calpain is quite complex and has not yet been fully elucidated. Unlike many proteases involved in protein degradation, calpain activity triggers a number of cellular modifications such as enzyme modulation (e.g. phospholipase C, calcineurin, PKC), and the conformational change of structural proteins (e.g. microtubule-associated proteins, lens proteins), membrane-associated proteins (e.g. receptors, ion channels, adhesion molecules), transcription factors (e.g. Fos, Jun), and more (Suzuki et al. 1995). It is of particular interest to Alzheimer disease that APP itself was identified as a calpain substrate in activated platelets (Li et al.1995). Moreover, calpain was found to be activated in Alzheimer brain compared to control brains, and this activation was more pronounced in the brain regions most affected by the disease (Nixon et al.1994; Saito et al.1993). The present invention is the discovery of a new interaction between the small (regulatory) subunit of calpain and acetylcholine esterase (AChE). The bait used in the search was aa 31 to 137 of AChE, and the prey was aa 1 to 268 of the small calpain subunit (full-length). Because cholinergic neurons are particularly affected in Alzheimer, the interaction between a calcium-activated protease and a cholinergic-specific enzyme allows the elaboration of an attractive model: a change in APP 30 metabolism (due for instance to mutations in APP or the presenilins) results in a disruption of calcium homeostasis which will alter calpain activity and trigger additional downstream modifications. These can include further alterations of APP metabolism as well as abnormal activation of AChE. Eventually, this cascade of events could result in amyloid accumulation and acetylcholine depletion. It is also important to note that calpain is essential for LTP (long term potentiation, the biochemical substrate of memory) in the hippocampus, the most severely affected brain area in AD (Denny et al.1990; Muller et al.1995). Thus, an interaction loop between APP and calpain (through calcium homeostasis) could lead independently to the cholinergic system (interaction with AChE) and memory (modulation of LTP). This is not surprising, since memory is known to be mediated in large part by hippocampal cholinergic neurons. Finally, The involvement of calpain in AD is also supported by recent reports of interactions between calpain and the presenilins (Steiner et al. 1998; Shinozaki et al. 1998). In summary, calpain is a protease that plays a crucial role in normal neuronal and synaptic functions, and interacts with major proteins involved in Alzheimer's (ACHE, APP, the presenilins). Calpain levels and activity show profound alterations in the brain of Alzheimer's patients. Therefore, modulation of calpain activity and/or its interaction pattern with other proteins is a promising new avenue for new drugs against Alzheimer's disease.

TABLE 10
Protein Complexes of AChE-KIAA0436 Interaction

Acetylcholine esterase (AChE) and KIAA0436
A fragment of AChE and KIAA0436
AChE and a fragment of KIAA0436
A fragment of AChE and a fragment of KIAA0436

The KIAA0436 protein was identified as an AChE interactor using two different AChE baits. We found that the KIAA0436 interacts with two non-overlapping domains of AChE, from aa 31 to 136, and from aa 266 to 354. The GenBank entry for KIAA0436 refers to the sequence as partial, probably because no stop codon was found upstream of the putative ATG initiation codon. However, our data suggest that this ATG may indeed be the correct initiation codon. First, Northern data show that the KIAA0436 protein is encoded by a 4.6 kb message, which is the same length as the GenBank entry. Thus, the GenBank sequence must be close to complete. Second, our 5′ RACE experiments identified only about 50 nucleotides upstream of the GenBank sequence, and a few of these sequences contained an in-frame stop codon upstream of the first ATG. Finally, the putative ATG initiation codon is in a good Kozak environment, with an A in position −3 and a G in position +4. Therefore, since this ATG is the first initiation codon in the sequence and is in a good Kozak environment, we consider it as the authentic initiation codon for the KIAA0436 protein. The KIAA is thus 638 aa long (and not 689 as reported in GenBank). The region of KIAA0436 that interacts with both ACHE baits is from aa 246 to 638 and contains a domain similar to prolyloligopeptidase from aa 397 to 475. The KIAA0436 protein is thus a novel protease that interacts with ACHE. The message for KIAA0436 is found at high levels in brain, medium levels in heart, low levels in kidney and pancreas, and undetected in placenta, lungs, liver, and skeletal muscle. In summary, we have identified a novel protease expressed preferentially in brain, and which interacts with AChE. As proteolytic events are known to be severely altered in Alzheimer brains, this protein is a promising new target candidate for drug discovery.

TABLE 11
Protein Complexes of AChE-α-Endosulfine Interaction
Acetylcholine esterase (AChE) and (APP695) and α-endosulfine
A fragment of AChE and α-endosulfine
AChE and a fragment of α-endosulfine
A fragment of AChE and a fragment of α-endosulfine

The small α-endosulfine protein (about 13 kDa) is 76% identical and 84% similar to the cAMP-regulated phosphoprotein 19 (Virsolvy-Vergine et al. 1996), which is a protein kinase A (PKA) substrate (Horiuchi et al.1990; Girault et al.1990), as is endosulfine itself (Roch et al.1997). Endosulfine is an endogenous ligand for SUR1, the type-1 sulfonylurea receptor. SUR1 is the regulatory subunit of ATP-sensitive inward rectifying potassium channels (KATP channels), while the channel-forming unit belongs to the Kir6.x family (Inagaki et al.1997). A major role of these channels is to link the metabolic state of the cell to its membrane potential: K_{ATP} channels close upon binding intracellular ATP to depolarize the cell and open when ATP concentrations return to resting levels (Ashcroft, 1988; Aguilar-Bryan et al. 1995; Inagaki et al. 1995; Freedman, Lin, 1996). These channels are involved in events such as insulin secretion from pancreatic p cells, ischemia responses in cardiac and cerebral tissues, and regulation of vascular smooth muscle tone. The activity of these channels in pancreatic β cells, where they play a crucial role in the secretion of insulin (Bryan, Aguilar-Bryan, 1997), has been extensively studied: following an elevation of blood glucose levels, the intracellular concentration of ATP in pancreatic β cells rise, resulting in channel closure and cell depolarization. This allows Ca²⁺ ions to enter the cell through voltage-sensitive Ca²⁺ channels, which will trigger the fusion of insulin secretory vesicles with the plasma membrane and release of insulin. In neurons, the same mechanisms involving K_{ATP} channels (linking the metabolic state of the cell to its membrane potential) control neurotransmitter release. It was shown in the pancreas that when endosulfine binds SUR1, the channel shuts down, thus stimulating insulin release. It is therefore believed that in the brain, endosulfine binding to SURI would also shut down K_{ATP} channels, leading to depolarization, Ca²⁺ entry, vesicle fusion, and release of the vesicular content into the synaptic cleft. In brief, endosulfine is a small protein regulating processes like neurotransmitter release and secretion of other factors from polarize cells. Its interaction with AChE suggests that endosulfine may be expressed in cholinergic neurons, and may control the release of acetylcholine and/or AChE from synaptic terminals.

TABLE 12
Protein Complexes of AChE-GIPC Interaction
Acetylcholine esterase (AChE) and GIPC (RGS-GAIP interacting protein)
A fragment of APP695 and GIPC
APP695 and a fragment of GIPC
A fragment of APP695 and a fragment of GIPC

An interaction between ACHE and δ-catenin was identified as described below. Because δ-catenin interacts with PS1 (Zhou et al.1997b; Tanahashi, Tabira, 1999; Kosik, 1998) and because of the involvement of the cholinergic is system in AD (Gooch, Stennett, 1996; Alvarez et al. 1998; Inestrosa, Alarcon, 1998), this novel interaction puts δ-catenin and AChE interactors in the heart of Alzheimer pathology.

GIPC was found to interact with AchE and δ-catenin. This common ACHE and δ-catenin interactor is reported to contain a PDZ domain (De Vries et al.1998b), and the C-terminus of δ-catenin (present in our bait) appears to be a PDZ-binding domain. The same study reports that GIPC interacts with the C-terminus of a protein called RGS-GAIP, which is a GTPase activating protein for Gαi heterotrimeric G-proteins (De Vries et al. 1998b). GAIP was recently shown to be located on clathrin-coated vesicles (De Vries et al.1998a). Therefore, when considering the interactions between PS1 and δ-catenin (Zhou et al.1997b; Tanahashi, Tabira, 1999; Kosik, 1998) and between PS1 and rab11 as described above, the pieces of a complex puzzle come together: the GAIP-GIPC complex (involved in GTPase activation) could be brought into the proximity of a potential GTPase target like rab11a through interactions of GIPC with δ-catenin, δ-catenin with PS1, and PS1 with rab11a. It is also remarkable that both GAIP and PS1 have been located in clathrin-coated vesicles (De Vries et al. 1998a; Efthimiopoulos et al. 1998), and that we found δ-catenin to interact with clathrin. When PS1 was first discovered (and first named S182), its physiological function was unknown, although it was speculated that PS1 was involved in protein trafficking (Hardy, 1997). The pattern of interactions that is now taking shape around PS1 fully supports this original speculation. The interactions of PS1 and δ-catenin with rab11a, GIPC, and clathrin suggest a crucial role in the control of intracellular vesicle trafficking. Because APP is also found in rab11-positive clathrin-coated vesicles, the control of vesicle trafficking is important in determining the ultimate fate of the APP molecules leading to Aβ release or secretion of neurotrophic/protective sAPP. It should also be pointed out that a mouse homolog of GIPC was cloned and described in GenBank. In the first entry, the mouse GIPC is named synactin (accession number AF104358), a protein that interacts with syndecan, a cell surface heparin-sulfate proteoglycan that links the cytoskeleton to the extracellular matrix. In another entry, mouse GIPC is called Semcap1 (accession number AF061263), which stands for “semaphorin F cytoplasmic domain associated protein 1”. Thus, GIPC is also thought to interact with semaphorin F, and therefore, it is possibly involved in axonal outgrowth and guidance.

The interaction pattern of GIPC puts it at the heart of the control of vesicle trafficking and membrane fusion, with direct consequences on the metabolism of proteins such as APP, PS1, δ-catenin, and AChE.

TABLE 13
Protein Complexes of AChE-δ-Catenin Interaction
Acetylcholine esterase (AChE) and δ-Catenin
A fragment of AChE and δ-Catenin
AChE and a fragment of δ-Catenin
A fragment of AChE and a fragment of δ-Catenin

TABLE 14
Protein Complexes of δ-Catenin-GIPC Interaction
δ-Catenin and GIPC (RGS-GAIP interacting protein)
A fragment of δ-Catenin and GIPC
δ-Catenin and a fragment of GIPC
A fragment of δ-Catenin and a fragment of GIPC

TABLE 15
Protein Complexes of δ-Catenin-Clathrin Interaction
δ-Catenin and Clathrin
A fragment of δ-Catenin and Clathrin
δ-Catenin and a fragment of Clathrin
A fragment of δ-Catenin and a fragment of Clathrin

TABLE 16
Protein Complexes of δ-Catenin-Plakophilin 2 Interaction
δ-Catenin and Plakophilin 2
A fragment of δ-Catenin and Plakophilin 2
δ-Catenin and a fragment of Plakophilin 2
A fragment of δ-Catenin and a fragment of Plakophilin 2

TABLE 17
Protein Complexes of δ-Catenin-Bcr Interaction
δ-Catenin and Bcr
A fragment of δ-Catenin and Bcr
δ-Catenin and a fragment of Bcr
A fragment of δ-Catenin and a fragment of Bcr

TABLE 18
Protein Complexes of δ-Catenin-14-3-3-beta Interaction
δ-Catenin and 14-3-3-beta
A fragment of δ-Catenin and 14-3-3-beta
δ-Catenin and a fragment of 14-3-3-beta
A fragment of δ-Catenin and a fragment of 14-3-3-beta

TABLE 19
Protein Complexes of δ-Catenin-14-3-3-zeta Interaction
δ-Catenin and 14-3-3-zeta
A fragment of δ-Catenin and 14-3-3-zeta
δ-Catenin and a fragment of 14-3-3-zeta
A fragment of δ-Catenin and a fragment of 14-3-3-zeta

TABLE 20
Protein Complexes of δ-Catenin-FAK2 Interaction
δ-Catenin and Focal adhesion kinase 2 (FAK2)
A fragment of δ-Catenin and FAK2
δ-Catenin and a fragment of FAK2
A fragment of δ-Catenin and a fragment of FAK2

TABLE 21
Protein Complexes of δ-Catenin-Eps8 Interaction

δ-Catenin and EGF receptor kinase substrate 8 (Eps8)
A fragment of δ-Catenin and Eps8
δ-Catenin and a fragment of Eps8
A fragment of δ-Catenin and a fragment of Eps8

TABLE 22
Protein Complexes of δ-Catenin-KIAA0443 Interaction

δ-Catenin and KIAA0443
A fragment of δ-Catenin and KIAA0443
δ-C atenin and a fragment of KIAA0443
A fragment of δ-Catenin and a fragment of KIAA0443

TABLE 23
Protein Complexes of NACP-δ-Catenin Interaction

Non-Aβcomponent of amyloid plaques precursor, 695 isoform
(NACP) and δ-Catenin
A fragment of NACP and δ-Catenin
NACP and a fragment of δ-Catenin
A fragment of NACP and a fragment of δ-Catenin

TABLE 24
Protein Complexes of ERAB-δ-Catenin Interaction

ERAB and δ-Catenin
A fragment of ERAB and δ-Catenin
ERAB and a fragment of a-Catenin
A fragment of ERAB and a fragment of δ-Catenin

TABLE 25
Protein Complexes of Bcl2-δ-Catenin Interaction

Bcl2and δ-Catenin
A fragment of Bcl2 and δ-Catenin
Bcl2 and a fragment of δ-Catenin
A fragment of Bcl2 and a fragment of δ-Catenin

APP metabolism is a critical event in the pathogenesis of Alzheimer's, because it leads to the release of either toxic (Aβ) or trophic (sAPP) metabolites (Cummings et al. 1998; Roch, Puttfarcken, 1996). In this respect, it is very important to identify proteins involved in the intracellular trafficking of APP. Genetic evidence suggest that PS1 and PS2 participate in this process, which may be perturbed by Alzheimer-causing mutations in APP or the presenilins (Hardy, 1997; Selkoe, 1998). The finding that PS1 interacts with rab11 (provisional patent application Ser. No. 60/113,534, filed Dec. 22, 1998, incorporated herein by reference) also supports a role for PS1 in the control of APP trafficking.

The family of proteins containing an armadillo domain includes plakophilin 1 and 2, neural-specific plakophilin (also known as δ-catenin), α-, β-, and δ-catenin. These proteins combine structural roles (as cell-contact and cytoskeleton-associated proteins) as well as signaling functions (by generating and transducing signals affecting gene expression) (Hatzfeld, 1999). Recently, PS1 was found to interact with several members of the armadillo family, including β-, δ-, and γ-catenin (Zhou et al. 1997b; Yu et al. 1998; Murayama et al. 1998; Zhou et al. 1997a; Tanahashi, Tabira, 1999; Kosik, 1998). While the significance of the γ-catenin interaction is not clear, it was suggested that the interaction between PS1 and β-catenin is important for neuronal survival (Zhang et al. 1998). To date, the interaction between PS1 and δ-catenin has not yielded many clues to AD pathogenesis, however the brain-specific expression pattern of δ-catenin suggests an important function in neuronal cells, which could be disrupted by mutations in the presenilins. In addition, an interaction between acetylcholine esterase (AChE) and δ-catenin was identified in a yeast two-hybrid search, using overlapping AChE baits, from aa 63 to 534, from aa 355 to 614, and from aa 355 to 517 (the smallest bait, which includes the δ-catenin binding domain). Because δ-catenin interacts with PS1 (Zhou et al.1997b; Tanahashi, Tabira, 1999; Kosik, 1998) and because of the involvement of the cholinergic is system in AD (Gooch, Stennett, 1996; Alvarez et al. 1998; Inestrosa, Alarcon, 1998), this novel interaction puts δ-catenin and ACHE interactors in the heart of Alzheimer pathology. In other words, all δ-catenin interactors are potentially involved in Alzheimer's. A structural role for δ-catenin is suggested by the following discovery: using a domain from aa 516 to 833 of δ-catenin as a bait in a yeast two-hybrid search, we found the heavy chain of clathrin (also known as KlAA0034) as an interactor. The C-terminal fragment of APP contains the YENPTY consensus sequence of proteins that are recycled from the plasma membrane into clathrin-coated pits, and from there to endosomes (McLoughlin, Miller, 1996; Zambrano et al. 1997; Russo et al. 1998). Moreover, a recent study showed that C- and N-terminal proteolytic fragment of PS1 are enriched in clathrin-coated vesicles of the somato-dendritic neuronal compartment (Efthimiopoulos et al. 1998). The authors claimed that “PS1 proteolytic fragments are targeted to specific populations of neuronal vesicles where they may regulate vesicular function”. Thus, the new interaction pattern that is emerging suggests that the δ-catenin-PS1 complex plays a central role in the intracellular trafficking of APP, through interactions with clathrin and rab11. This statement is further supported by the discovery of other interactions involving δ-catenin, described below.

Cell-cell adhesion plays important roles in development, tissue morphogenesis, and in the regulation of cell migration and proliferation, all crucial events in brain development and function. Desmosomes are adhesive intercellular junctions that anchor the intermediate filament network to the plasma membrane. By functioning both as an adhesive complex and as a cell-surface attachment site for intermediate filaments, desmosomes integrate the intermediate filament cytoskeleton between cells and play an important role in maintaining tissue integrity. Using a domain of δ-catenin from aa 516 to 833 in a yeast two-hybrid search, we identified plakophilin 2 as a prey. Like δ-catenin, plakophilin 2 is a member of the armadillo family. Specifically, plakophilin 2 has been found both in desmosomes and in the nucleus (Mertens et al.1996), suggesting a dual cellular role. The interaction between δ-catenin (a brain specific armadillo protein) and plakophilin 2 suggests that δ-catenin and its interactors (including PS1) are involved in functions such as cell adhesion and control of gene expression. In this respect, it is worth noting that APP can mediate cell adhesion (Breen et al. 1991), and has also been found associated with nuclear proteins and transcription factors (Russo et al.1998), hence a potential role in transcriptional regulation.

Recently, we found δ-catenin as a prey in a yeast two-hybrid search, using NACP as a bait. NAC (Non-Aβ Component of amyloid plaques) is a peptide of 35 residues originally isolated from amyloid material in Alzheimer cortex (Ueda et al.1993). Cloning of a cDNA coding for NAC revealed that NAC is generated by proteolytic cleavage of a larger protein, NACP (NAC precursor) (Ueda et al. 1993). It is interesting that the two major components of the plaques (Aβ and NAC) are both generated by cleavage of a precursor protein (APP and NACP). Further studies showed that the NAC peptide is itself amyloidogenic (it self-aggregates into amyloid material) and that it binds Aβ and stimulates its aggregation (Yoshimoto et al. 1995; Iwai et al.1995b). In addition, NACP was identified as a presynaptic protein in the central nervous system, suggesting a role in synaptic function (Iwai et al.1995a). Thus, cleavage of NACP into NAC results in the release of an amyloidogenic fragment and may independently impair synaptic function. The similarity with APP/Aβ is again striking. Indeed, another study suggested that there is a connection between the metabolism of presynaptic proteins and amyloid formation (Masliah et al. 1996). In this respect, it should also be noted that ApoE4 binding to NAC is twice as strong as that of ApoE3 (Olesen et al. 1997), and the presence of the E4 allele has been identified as a risk factor for AD (Hardy, 1995; Strittmatter, Roses, 1995; Falduto, LaDu, 1996). Recently, mutations in NACP have been found to co-segregate with early-onset familial Parkinson's disease (Polymeropoulos et al. 1997). Furthermore, these mutations were shown to disrupt NACP binding to brain vesicles involved in fast axonal transport (Jensen et al., 1998). As APP is known to undergo fast axonal transport (Koo et al., 1990), the δ-catenin-NACP connection again brings δ-catenin right into the intracellular trafficking of APP, at the heart of AD pathogenesis.

The mechanism of Aβ toxicity has always been controversial (Iversen et al., 1995; Manelli, Puttfarcken, 1995; Gillardon et al., 1996; Behl et al., 1992; Weiss et al., 1994; Octave, 1995; Furukawa et al., 1996b; Schubert, 1997). Reports of neuronal apoptosis have been contradicted by studies showing necrosis was the cause of cell death (Loo et al.1993; Behl et al. 1994; Bancher et al. 1997; Schubert, 1997). In any event, it is clear that events such as generation of free radicals, lipid peroxidation, and disruption of calcium homeostasis play a major role in Aβ toxicity (Weiss et al.1994; Abe, Kimura, 1996; Mark et al.1997; Kruman et al.1997). To elucidate this phenomenon, investigators used the yeast two-hybrid system to look for proteins that interact with the Aβ peptide and could mediate its toxicity. A novel protein named ERAB was identified (Yan et al. 1997), which later turned out to be identical to a 3-hydroxyacyl-CoA dehydrogenase (He et al. 1998). The original report also claimed that ERAB mediates Aβ toxicity (Yan et al. 1997), and a recent study showed that it does so by generating toxic adlehydes from alcohol (Yan et al. 1999). To gain more information about ERAB, we used the full-length protein as a bait in a yeast two-hybrid search and found δ-catenin as a prey. This interaction, as the δ-catenin-NACP interaction described above, brings δ-catenin in the heart of APP metabolism. Also, the interactions between ERAB and Aβ (a proteolytic product of APP), between ERAB and δ-catenin, and between δ-catenin and PS-1 generate a possible biochemical link between PS-1 and APP, which could explain how the FAD mutations in PS1 can alter APP metabolism.

Thus, the five novel interactions we identified so far and that involve δ-catenin (with AChE, ERAB, NACP, clathrin, and plakophilin 2) put it at the crossroads of biochemical and cellular events involved in AD pathogenesis. Although δ-catenin by itself may not be a suitable drug target, drugs that would alter its interaction pattern could be of interest for Alzheimer's disease. Likewise, other δ-catenin interactors could become attractive drug targets, precisely because of the intimate connection between δ-catenin and AD pathogenesis.

The product of the bcl-2 proto-oncogene is a mitochondrial protein that was shown to block neuronal apoptosis (Hockenbery et al. 1990). The anti-apoptotic activity of bcl-2 is quite relevant to Alzheimer's in the light of two recent studies that showed that bcl-2 blocks neuronal death induced by Aβ in transgenic mice (Cribbs et al. 1994), or by FAD-associated PS1 mutations in transfected cells (Guo et al.1997). However, a direct biochemical link between bcl-2 and Alzheimer's related protein has not been shown yet. Using a domain of bcl-2 from aa 1 to 75 in a yeast two-hybrid search, we found a domain from aa 690 to 1225 of δ-catenin as a prey. This interaction generates a link between PS1 and bcl-2 and might explain the anti-apoptotic activity of wild-type PS1, and why FAD associated mutations in PS1 activate neuronal apoptosis (Guo et al.1997; Kim, Tanzi, 1997; Kovacs, Tanzi, 1998; Tesco et al.1998). In this respect, drugs that modulate the interaction between δ-catenin and PS1 and between δ-catenin and bcl-2 might help prevent neuronal apoptosis as observed in the brain of AD patients.

Using two δ-catenin domains as baits in yeast two-hybrid searches, from aa 516 to 833 and from aa 1006 to 1158, we found respectively the break point cluster (Bcr) protein and the 14-3-3β protein as preys. Interestingly, these two proteins are known to interact with each other (Braselmann, McCormick, 1995). Bcr is a GTP-binding protein which activates GTPases of the Ras family (Diekmann et al. 1995), and participates in the chromosomal translocation with the c-Abl oncogene to generate the Bcr-Abl oncogene responsible for several forms of leukemia (Warmuth et al. 1999). In addition, Bcr and c-Abl were shown to interact directly with each other (Pendergast et al.1991). The GTPase activating function of Bcr is interesting in the light of the PS1-rab11 interaction (provisional patent application Ser. No. 60/113,534, filed Dec. 22, 1998, incorporated herein by reference). The rab11 protein is also a GTPase, involved in intracellular vesicle trafficking and membrane fusion, and expressed in the CNS (Ullrich et al.1996; Sheehan et al.1996; Chen et al.1998). Thus, the δ-catenin-Bcr complex could modulate vesicle trafficking though interactions with PS1 and rab11. FAD associated mutations in PS1 could alter disrupt these interaction and alter the proper trafficking machinery, leading to the production of toxic metabolites like Aβ. The 14-3-3β protein is a well known modulator of protein kinase C (PKC) and is expressed at high levels in the CNS (Skoulakis, Davis, 1998; Aitken et al.1995). PKC activity is an critical factor regulating α-secretion of APP (Govoni et al.1996; Rossner et al.1998; Jin, Saitoh, 1995). Thus, as PS1 interacts with δ-catenin and δ-catenin interacts with Bcr and 14-3-3β (which also interact with each other), FAD-associated mutations in PS-1 could influence the stability of the complex formed by δ-catenin, bcr, and 14-3-3β, which in turn could affect PKC activity and α-secretion of APP. A similar model has recently been proposed for the effect of FAD-associated mutations in PS1 that could destabilize a β-catenin complex and trigger neuronal apoptosis (Zhang et al. 1998). Therefore, drugs that would modulate the interactions of δ-catenin with Bcr and/or with 14-3-3β could control α-secretase activity and the eventual generation of the trophic secreted form of APP or the toxic Aβ peptide. Finally, another important connection can be made between the δ-catenin-14-3-3β pathway and the PS1-FKBP25 pathway. FKBP25 is a protein from the immunophilin family and is involved in the neurotrophic effects of immunosuppressant drugs such as FK506 and rapamycin (Snyder et al.1998; Steiner et al.1997a; Steiner et al.1997b). While the FK506 effects are mediated by the calcium-activated phosphatase calcineurin (Snyder et al. 1998), rapamycin effects are transduced by the TOR kinase (Chiu et al.1994; Lorenz, Heitman, 1995). Although FKBP25 binds FK506, it has a much higher affinity for rapamycin (Galat et al. 1992), suggesting that FKBP25 signals through the TOR kinase system. Recently, it was shown that the rapamycin signaling pathway uses 14-3-3β (Bertram et al.1998). Thus, the neurotrophic effect elicited by FKBP25 (a PS1 interactor) are likely to be mediated by 14-3-3 β (a δ-catenin interactor). Again, it is possible that FAD-associated mutations in PS1 could disrupt its interaction with 6-catenin, and thus impair the 14-3-3β-mediated neurotrophic effect of FKBP25.

The same yeast two search using the domain of δ-catenin from aa 1006 to 1158 as a bait also returned the protein 14-3-3ζ as a prey, which is also a PKC modulator (Aitken et al. 1995) and which is 87% identical (93% similar) to 14-3-3β. It is not known whether 14-3-3ζ interacts with Bcr, as 14-3-3β does. In any case, its PKC modulating activity and its interaction with δ-catenin also make possible for the PS1-δ-catenin complex to control α-secretase activity and thus the production of the trophic secreted form of APP or the toxic Aβ peptide.

The same yeast two search using the domain of δ-catenin from aa 1006 to 1158 as a bait also returned the focal adhesion kinase 2 (FAK2) as a prey, also called proline-rich tyrosine kinase 2 (PYK2) or cell adhesion kinase β (CAKβ). Focal adhesion kinases (FAKs) form a special subfamily of cytoplasmic protein tyrosine kinases (PTKs). In contrast to other non-receptor PTKs, FAKs do not contain SH2 or SH3 domains, but have a carboxy-terminal proline-rich domain which is important for protein-protein interactions (Schaller, 1997; Schaller, Parsons, 1994; Parsons et al.1994). FAK2 is expressed at highest levels in brain, at medium levels in kidney, lung, and thymus, and at low levels in spleen and lymphocytes (Avraham et al.1995). In brain, FAK2 is found at highest levels in the hippocampus and amygdala (Avraham et al.1995), two areas severely affected in Alzheimer's disease. FAK2 is thought to participate in signal transduction mechanisms elicited by cell-to-cell contacts (Sasaki et al. 1995). It is involved in the calcium-induced regulation of ion channels, and it is activated by the elevation of intracellular calcium concentration following the activation of G protein-coupled receptors (GPCRs) that signal though Gαq and the phospholipase C (PLC) pathway (Yu et al.1996). Thus, FAK2 is an important intermediate signaling molecule between GPCRs activated by neuropeptides or neurotransmitters and downstream signals that modulate the neuronal activity (channel activation, membrane depolarization). Such a link between intracellular calcium levels, tyrosine phosphorylation, and neuronal activity is clearly important for neuronal survival and synaptic plasticity (Siciliano et al. 1996). The interaction of FAK2 with δ-catenin and its high levels of expression in hippocampus and amygdala suggest that a disruption of its activity may be related to neuronal death in AD. Drugs that would modulate FAK2 activity or its interaction with δ-catenin may thus prove beneficial.

Using a domain of δ-catenin from aa 516 to 833, we identified the EGF receptor kinase substrate 8 (Eps8) as a prey. This is a protein of 822 amino acids which is an intracellular substrate for a several receptors with tyrosine kinase activity as well as for non-receptor kinase. Upon binding to the EGF receptor, it enhances mitogenic signals mediated by EGF (Fazioli et al.1993; Wong et al.1994). Eps8 is thought to play an essential function in cell growth regulation and in the reorganization of the cytoskeleton, perhaps acting as a docking site for other signaling molecules (Provenzano et al. 1998). In this respect, δ-catenin could be a bridge between Eps8 and FAK2 or another tyrosine kinase. As Eps8 is associated with cell division, abnormal signaling through Eps8 leading to mitosis could trigger apoptosis in post-mitotic cells such as neurons. Thus, drugs that modulate Eps8 could enhance neuronal survival.

Using a domain of δ-catenin from aa 1006 to 1158, we identified the KIAA0443 protein as a prey. This is a novel protein for which a cDNA was randomly cloned out of a human brain library (Ishikawa et al. 1997). Searching for motifs and patterns in the KIAA0443 amino acid sequence revealed the presence of an ATP/GTP binding domain. Therefore, it's possible that KIAA0443 is a GTP or ATP exchange factor that functions together with another δ-catenin interactor such as Bcr or FAK2, or with a PS1 interactor such as rab11. We also identified several lipocalin signature domains in KIAA0443, which suggest that this protein may be involved in the transport of small hydrophobic molecules. Although the biological function of KIAA0443 is not clear at this point, its interaction with δ-catenin, a brain-specific protein, suggests that it is involved in some kind of brain-specific function. Drugs that modulate the δ-catenin-KIAA0443 interaction could thus influence neuronal and synaptic functions.

TABLE 26
Protein Compexes of PS1 -α-enolase Interaction

Presenilin 1 (PS1) and α-enolase
A fragment of PS1 and α-enolase
PS1 and a fragment of α-enolase
A fragment of PS1 and a fragment of α-enolase

TABLE 27
Protein Complexes of Axin-Citrate Synthase Interaction

Axin and Citrate Synthase
A fragment of Axin and Citrate Synthase
Axin and a fragment of Citrate Synthase
A fragment of Axin and a fragment of Citrate Synthase

TABLE 28
Protein Complexes of Axin-Aldolase C Interaction

Axin and Aldolase C
A fragment of Axin and Aldolase C
Axin and a fragment of Aldolase C
A fragment of Axin and a fragment of Aldolase C

TABLE 29
Protein Complexes of Axin-Creatine kinase B Interaction

Axin and Creatine kinase B
A fragment of Axin and Creatine kinase B
Axin and a fragment of Creatine kinase B
A fragment of Axin and a fragment of Creatine kinase B

TABLE 30
Protein Complexes of Axin-Neurogranin Interaction

Axin and Neurogranin
A fragment of Axin and Neurogranin
Axin and a fragment of Neurogranin
A fragment of Axin and a fragment of Neurogranin

TABLE 31
Protein Complexes of Axin-Rab3A Interaction

Axin and Rab3A
A fragment of Axin and Rab3A
Axin and a fragment of Rab3A
A fragment of Axin and a fragment of Rab3A

TABLE 32
Protein Complexes of Axin-AOP-1 Interaction

Axin and Anti-oxidant mitochondrial protein (AOP-1)
A fragment of Axin and AOP-1
Axin and a fragment of AOP-1
A fragment of Axin and a fragment of AOP-1

TABLE 33
Protein Complexes of Axin-SMN1 Interaction

Axin and SMN1
A fragment of Axin and SMN1
Axin and a fragment of SMN1
A fragment of Axin and a fragment of SMN1

TABLE 34
Protein Complexes of Axin-SRp30c Interaction

Axin and SRp3Oc
A fragment of Axin and SRp30c
Axin and a fragment of SRp30c
A fragment of Axin and a fragment of SRp30c

TABLE 35
Protein Complexes of PS1-LSF Interaction

Presenilin 1 (PS1) and LSF
A fragment of PS1 and LSF
PS1 and a fragment of LSF
A fragment of PS1 and a fragment of LSF

TABLE 36
Protein Complexes of LSF-APP Interaction

LSF and Amyloid β protein precursor (APP)
A fragment of LSF and APP
LSF and a fragment of APP
A fragment of LSF and a fragment of APP

TABLE 37
Protein Complexes of LSF-4F5s Interaction

LSF and 4F5s
A fragment of LSF and 4F5s
LSF and a fragment of 4F5s
A fragment of LSF and a fragment of 4F5s

There is a growing body of evidence that disruption of energy metabolism is an important factor in neurodegenerative disorders, including Alzheimer's Disease (Beal, 1998; Nagy et al.1999; Rapoport et al.1996). Mitochondrial dysfunctions result in low ATP levels and production of free oxiradicals that are extremely toxic to neurons (Simonian, Coyle, 1996; Beal, 1996). To gain insight into the involvement of the mitochondrial machinery in AD pathogenesis, we used Alzheimer related proteins as baits in yeast two-hybrid searches and looked for interactors that are either mitochondrial proteins, or somehow involved in energy metabolism.

First, we found an interaction between PS-1 and α-enolase, a glycolytic enzyme which transforms 2-phosphoglycerate into phosphoenol pyruvate, and is thus directly involved in energy production. Next, the enzymes citrate synthase and aldolase C were found to interact with axin. Aldolase is active as a homotetramer, involved in glycolysis (it cleaves fructose bi-phosphate into dihydroxyacetone phosphate and glyceraldehyde 3-phosphate). The 3 isoforms A, B, and C are found respectively in muscle, liver, and brain. Citrate synthase is the enzyme catalyzing the first step of the Krebs cycle, the condensation of oxaloacetate and acetyl-CoA into citrate, with release of CoA and energy (−7.7 kcal/mol) production. Unlike aldolase and α-enolase (cytosolic), citrate synthase is located in the mitochondrial matrix. We also found an interaction between axin and creatine kinase B. This is a well characterized cytosolic enzyme involved in energy metabolism, and is likely to be very important for an organ like brain where the demand for energy fluctuates rapidly and over a large range. Creatine kinase exists in two cytosolic isoforms called M and B, plus two mitochondrial isoforms. The cytosolic enzyme is active either as homo- or heterodimers. The MM enzyme is found in heart and skeletal muscle, the MB enzyme mostly in heart, and the BB enzyme in many tissues, mainly brain.

In addition, we identified an interaction between axin and neurogranin. This is a small (78 residues) protein which belongs to the calpacitin family (together with GAP-43 and PEP-19). While GAP-43 is found in the axonal compartment, neurogranin is associated with post-synaptic membranes (Gerendasy, Sutcliffe, 1997). It is involved in the development of dendritic spines, LTP, LTD, learning and memory (Gerendasy, Sutcliffe, 1997). Although its exact function is not clear yet, available models claim that neurogranin regulates the availability of calnodulin, and in turn, calmodulin regulates neurogranin's ability to amplify the mobilization of calcium in response to stimulation of metabotropic glutamate receptor. Neurogranin and GAP-43 release calmodulin rapidly in response to a large calcium influx, and slowly in response to a small influx. Therefore, these proteins act like a “calcium capacitor” (hence the name calpacitin). The amount of calcium that the system can handle (capacitance) is regulated by PKC phosphorylation of neurogranin (and GAP-43), which blocks its binding to calmodulin (Gerendasy, Sutcliffe, 1997). Therefore, the ratio of phosphorylated to non-phosphorylated neurogranin could control the LTP/LTD sliding threshold (together with calcium-calmodulin dependent kinase II). Most importantly, neurogranin has been reported to be associated with mitochondria, in order to couple energy production with dendritic spine formation and synaptic plasticity (Gerendasy, Sutcliffe, 1997). Finally, we also found interaction between axin with a thioredoxin-dependent peroxide reductase, an anti-oxidant mitochondrial protein (AOP-1). The anti-oxidant properties of this protein suggest that is might protect neurons role against oxidative insults, as the anti-oxidant vitamin E does (Behl et al.1992). In summary, using two neuronal proteins (axin and PS-1), one of which (PS1) being directly involved in AD, as baits in yeast two-hybrid searches, we have identified six important interactors. Four of these are enzymes involved in energy production (α-enolase, aldolase C, citrate synthase, and creatine kinase B), one is a protein involved in the formation of dendritic spines, LTP, and memory, and the last one is a known anti-oxidant protein. In the light of the well documented mitochondrial disorders associated with some neurodegenerative conditions (Beal, 1998; Nagy et al.1999), often involving the production of toxic oxiradical species (Busciglio, Yankner, 1995; Richardson et al.1996; Simonian, Coyle, 1996; Beal, 1996), these newly identified interactions open new promising therapeutic and diagnostic avenues.

We also found an interaction between axin and the small GTPase rab3A. Like rab11, this protein is involved in intracellular vesicle trafficking. Specifically, rab3A plays a major role in the trafficking of synaptic vesicles (Geppert, Sudhof, 1998) and thus, may regulate neurotransmitter release. Rab3A expression is reported to be brain specific, and essential for LTP of mossy fiber synapses in the hippocampus (Castillo et al. 1997), the most severely affected area in Alzheimer brains. This observation is crucial because LTP is known to be impaired in the hippocampus of mice transgenic for the carboxy-terminal region of APP (Nalbantoglu et al. 1997).

We also report interactions that are closely biologically related because 1) the baits (axin and LSF) are intimately involved in AD (through direct interactions with notorious Alzheimer proteins), and 2) because of the functional similarity of the preys. Axin was found to interact with two proteins involved in RNA metabolism, the splicing factors SRp30c and SMN1 (survival for motor neurons). These two proteins contain 221 and 294 amino acids, respectively and are part of the spliceosome complex (Screaton et al.1995; Pellizzoni et al.1998; Talbot et al.1997). The relevance of these interactions in an Alzheimer's perspective is that mutations in SMN1 cause a variety of autosomal recessive neurodegenerative disorders, including SMA (spinal muscular atrophy), that can be distinguished by the age of onset and the severity of the clinical features and are characterized by the degeneration of lower motor neurons, resulting paralysis (Lefebvre et al.1998; Lefebvre et al.1995). The outcome is often fatal. SMN1 is expressed in many regions of the central nervous system, including spinal cord, brainstem, cerebellum, thalamus, cortex (especially the layer V, most affected in AD patients) and hippocampus (also deeply affected in AD) (Bechade et al. 1999). A role for SMN1 in nucleocytoplasmic and dendritic transport has also been proposed (Bechade et al. 1999). In addition, the role of SMN1 in neuron survival is thought to be mediated by the anti-apoptotic protein bcl-2 (Lefebvre et al. 1998), which we found to interact with δ-catenin. Thus, axin interacts with 2 proteins involved in splicing, one of which is directly linked to the neuron survival and expressed in brain regions severely affected in AD. LSF is a transcription factor that was reported to interact with Fe65, a well known APP interactor (Zambrano et al. 1998). The relevance of this interaction remains obscure, although it has been proposed that the LSF/Fe65 complex could control APP trafficking and metabolism (Russo et al. 1998). Our own data reveal two important novel interactions: using PS1 as a bait in a yeast two-hybrid search, we found LSF as an interactor, and using LSF as a bait in a yeast two-hybrid searches, we found that it interacts directly with APP. Thus, LSF interacts directly with Fe65, APP, and PS1. This finding puts LSF and its interactors into the heart of AD pathogenesis. We also found that LSF interacts with a small protein (71 amino acids) called 4F5s. The function of this novel protein is totally unknown, but it was reported to be a potential genetic modifier of SNM1 (Scharf et al. 1998). It is unknown however, whether SMN1 and 4F5s interact directly.

In brief, we have identified a series of interactions (axin with SRp30c and SMN1, LSF with PS1, APP and 4F5s), which generates a network that brings the splicing factors SRp30c and SMN1 and the protein 4F5s into the heart of AD pathogenesis. Two of these proteins are directly involved in neuron survival, and the expression pattern of one of them is a good match with AD pathology. Thus, these newly identified interactions also open new promising therapeutic and diagnostic avenues against AD.

In view of the above description new pathways involving the major Alzheimer proteins can be elucidated. APP is the metabolic precursor of the Aβ peptide found in the core of neuritic amyloid plaques, and which is directly toxic to neurons. This pathway also release βsAPP, which shows a weak activity of neuronal survival, neurite outgrowth, synaptic maintenance and enhanced memory. However, another metabolic pathway (which is non-amyloidogenic) releases αsAPP, whose neurotrophic activity is much stronger than that of βsAPP. Mutations in PS1 are known to influence APP metabolism to produce Aβ42, the most toxic form of the Aβ peptide. Axin was found to interact with AOP-1, a mitochondrial enzyme which protects neurons against oxidative insults by free radicals. Axin also interacts with citrate synthase, aldolase C, and creatine kinase B, while PS1 interacts with α-enolase. These four enzymes are all involved in energy metabolism, the disruption of which is a known cause of neurodegeneration (Beal, 1998; Nagy et al.1999; Rapoport et al. 1996). Axin also interacts with rab3 and neurogranin, two proteins involved in the development of dendritic spines (a process that requires large amount of energy) and which are essential for LTP in the hippocampus.

APP and PS1 both interact with LSF, which also interacts with Fe65, which in turn interacts with APP. PS1 also interacts with δ-catenin, which in turn interacts with ERAB, an APP interactor. Thus, LSF, d-catenin, and their interactors are in the heart of AD pathogenesis. Axin interacts with SMN1 and SRp30c, two proteins involved in RNA metabolism. In addition, SMN1 is involved in neuronal survival, an activity which is mediated by bcl2, a δ-catenin interactor. In addition, the protein 4F5s is a genetic modifier of SMN1 and interacts with LSF.

The proteins disclosed in the present invention were found to interact with PS1, APP or other proteins involved in AD, in the yeast two-hybrid system. Because of the involvement of these proteins in AD, the proteins disclosed herein also participate in the pathogenesis of AD. Therefore, the present invention provides a list of uses of those proteins and DNA encoding those proteins for the development of diagnostic and therapeutic tools against AD. This list includes, but is not limited to, the following examples.

Two-hybrid System

The principles and methods of the yeast two-hybrid system have been described in detail elsewhere (e.g., Bartel and Fields, 1997; Bartel et al., 1993; Fields and Song, 1989; Chevray and Nathans, 1992). The following is a description of the use of this system to identify proteins that interact with a protein of interest, such as PS1.

The target protein is expressed in yeast as a fusion to the DNA-binding domain of the yeast Gal4p. DNA encoding the target protein or a fragment of this protein is amplified from cDNA by PCR or prepared from an available clone. The resulting DNA fragment is cloned by ligation or recombination into a DNA-binding domain vector (e.g., pGBT9, pGBT.C, pAS2-1) such that an in-frame fusion between the Gal4p and target protein sequences is created.

The target gene construct is introduced, by transformation, into a haploid yeast strain. A library of activation domain fusions (i.e., adult brain cDNA cloned into an activation domain vector) is introduced, by transformation into a haploid yeast strain of the opposite mating type. The yeast strain that carries the activation domain constructs contains one or more Gal4p-responsive reporter gene(s), whose expression can be monitored. Examples of some yeast reporter strains include Y190, PJ69, and CBY14a. An aliquot of yeast carrying the target gene construct is combined with an aliquot of yeast carrying the activation domain library. The two yeast strains mate to form diploid yeast and are plated on media that selects for expression of one or more Gal4p-responsive reporter genes. Colonies that arise after incubation are selected for further characterization.

The activation domain plasmid is isolated from each colony obtained in the two-hybrid search. The sequence of the insert in this construct is obtained by the dideoxy nucleotide chain termination method. Sequence information is used to identify the gene/protein encoded by the activation domain insert via analysis of the public nucleotide and protein databases. Interaction of the activation domain fusion with the target protein is confirmed by testing for the specificity of the interaction. The activation domain construct is co-transformed into a yeast reporter strain with either the original target protein construct or a variety of other DNA-binding domain constructs. Expression of the reporter genes in the presence of the target protein but not with other test proteins indicates that the interaction is genuine.

In addition to the yeast two-hybrid system, other genetic methodologies are available for the discovery or detection of protein-protein interactions. For example, a mammalian two-hybrid system is available commercially (Clontech, Inc.) that operates on the same principle as the yeast two-hybrid system. Instead of transforming a yeast reporter strain, plasmids encoding DNA-binding and activation domain fusions are transfected along with an appropriate reporter gene (e.g., lacZ) into a mammalian tissue culture cell line. Because transcription factors such as the Saccharomyces cerevisiae Gal4p are functional in a variety of different eukaryotic cell types, it would be expected that a two-hybrid assay could be performed in virtually any cell line of eukaryotic origin (e.g., insect cells (SF9), fungal cells, worm cells, etc.). Other genetic systems for the detection of protein-protein interactions include the so-called SOS recruitment system (Aronheim et al., 1997).

Protein-protein Interactions

Protein interactions are detected in various systems including the yeast two-hybrid system, affinity chromatography, co-immunoprecipitation, subcellular fractionation and isolation of large molecular complexes. Each of these method is well characterized and can be readily performed by one skilled in the art. See, e.g., U.S. Pat. Nos. 5,622,852 and 5,773,218, and PCT published application No. WO 97/27296, each of which are incorporated herein by reference.

The protein of interest can be produced in eukaryotic or prokaryotic systems. A cDNA encoding the desired protein is introduced in an appropriate expression vector and transfected in a host cell (which could be bacteria, yeast cells, insect cells, or mammalian cells). Purification of the expressed protein is achieved by conventional biochemical and immunochemical methods well known to those skilled in the art. The purified protein is then used for affinity chromatography studies: it is immobilized on a matrix and loaded on a column. Extracts from cultured cells or homogenized tissue samples are then loaded on the column in appropriate buffer, and non-binding proteins are eluted. After extensive washing, binding proteins or protein complexes are eluted using various methods such as a gradient of pH or a gradient of salt concentration. Eluted proteins can then be separated by two-dimensional gel electrophoresis, eluted from the gel, and identified by micro-sequencing. All of these methods are well known to those skilled in the art.

Purified proteins of interest can also be used to generate antibodies in rabbit, mouse, rat, chicken, goat, sheep, pig, guinea pig, bovine, and horse. The methods used for antibody generation and characterization are well known to those skilled in the art. Monoclonal antibodies are also generated by conventional techniques.

DNA molecules encoding proteins of interest can be inserted in the appropriate expression vector and used for transfection of eukaryotic cells such as bacteria, yeast, insect cells, or mammalian cells, following methods well known to those skilled in the art. Transfected cells expressing both proteins of interest are then lysed in appropriate conditions, one of the two proteins is immunoprecipitated using a specific antibody, and analyzed by polyacrylamide gel electrophoresis. The presence of the binding protein (co-immunoprecipitated) is detected by immunoblotting using an antibody directed against the other protein. Co-immunoprecipitation is a method well known to those skilled in the art.

Transfected eukaryotic cells or biological tissue samples can be homogenized and fractionated in appropriate conditions that will separate the different cellular components. Typically, cell lysates are run on sucrose gradients, or other materials that will separate cellular components based on size and density. Subcellular fractions are analyzed for the presence of proteins of interest with appropriate antibodies, using immunoblotting or immunoprecipitation methods. These methods are all well known to those skilled in the art.

Disruption of Protein-protein Interactions

It is conceivable that agents that disrupt protein-protein interactions can be beneficial in AD. Each of the methods described above for the detection of a positive protein-protein interaction can also be used to identify drugs that will disrupt said interaction. As an example, cells transfected with DNAs coding for proteins of interest can be treated with various drugs, and co-immunoprecipitations can be performed. Alternatively, a derivative of the yeast two-hybrid system, called the reverse yeast two-hybrid system (Lenna and Hannink, 1996), can be used, provided that the two proteins interact in the straight yeast two-hybrid system.

Modulation of Protein-protein Interactions

Since the interactions described herein are involved in the AD pathway, the identification of agents which are capable of modulating the interactions will provide agents which can be used to track AD or to use lead compounds for development of therapeutic agents. An agent may modulate expression of the genes of interacting proteins, thus affecting interaction of the proteins. Alternatively, the agent may modulate the interaction of the proteins. The agent may modulate the interaction of wild-type with wild-type proteins, wild-type with mutant proteins, or mutant with mutant proteins. Agents can be tested using transfected host cells, cell lines, cell models or animals, such as described herein, by techniques well known to those of ordinary skill in the art, such as disclosed in U.S. Pat. No. 5,622,852 and 5,773,218, and PCT published application No. WO 97/27296, each of which are incorporated herein by reference. The modulating effect of the agent can be treated in vivo or in vitro. Exemplary of a method to screen agents is to measure the effect that the agent has on the formation of the protein complex.

Mutation Screening

The proteins disclosed in the present invention interact with APP or PS1, the two major proteins involved in AD. Mutations in interacting proteins could also be involved in the development of AD, for example, through a modification of protein-protein interaction, or a modification of enzymatic activity, modification of receptor activity, or through an unknown mechanism. For exxample, the genes for APP and PS1 are known to contain mutations that cause AD in some families. Mutations in APP and PS1 interacting proteins could also be involved in the development of AD, for example, through a modification of protein-protein interaction, or a modification of enzymatic activity (e.g. the rotamase activity of FKBP25, or the GTPase activity of rab11, or the ubiquitin-like domain of BAT3), or through an unknown mechanism. Therefore, mutations can be found by sequencing the genes for the proteins of interest in AD patient and non-affected controls. A mutation in these genes, especially in that portion of the gene involved in protein interactions in the AD pathway, can be used as a diagnostic tool, and the mechanistic understanding the mutation provides can help develop a therapeutic tool.

Screening for At-risk Individuals

Individuals can be screened to identify those at risk by screening for mutations in the proteins disclosed herein and identified as described above. Alternatively, individuals can be screened by analyzing the ability of the proteins of said individual disclosed herein to form natural complexes. Techniques to detect the formation of complexes, including those described above, are known to those skilled in the art. Techniques and methods to detect mutations are well known to those skilled in the art.

Cellular Models of AD

A number of cellular models of AD have been generated and the use of these models is familiar to those skilled in the art. As an example, secretion of the Aβ peptide from cultured cells can be measured with appropriate antibodies. Likewise, the proportion of Aβ40 and Aβ42 can be readily determined. Neuron survival assays and neurite extension assays in the presence of various toxic agents (the Aβ peptide, free radicals, others) are also well known to those skilled in the art. Primary neuronal cultures or established neuronal cell lines can be transfected with expression vectors encoding the proteins of interest, either wild-type proteins or Alzheimer's-associated mutant proteins. The effect of these proteins on parameters relevant to AD (Aβ secretion, neuronal survival, neurite extension, or others) can be readily measured. Furthermore, these cellular systems can be used to screen drugs that will influence those parameters, and thus be potential therapeutic tools in AD. Alternatively, instead of transfecting the DNA encoding the protein of interest, the purified protein of interest can be added to the culture medium of the neurons, and the relevant parameters measured.

Animal Models

The DNA encoding the protein of interest can be used to create animals that overexpress said protein, with wild-type or mutant sequences (such animals are referred to as “transgenic”), or animals which do not express the native gene but express the gene of a second animal (referred to as “transplacement”), or animals that do not express said protein (referred to as “knock-out”). The knock-out animal may be an animal in which the gene is knocked out at a determined time. The generation of transgenic, transplacement and knock-out animals (normal and conditioned) uses methods well known to those skilled in the art.

In these animals, parameters relevant to AD can be measured. These include Aβ secretion in the cerebrospinal fluid, Aβ secretion from primary cultured cells, the neurite extension activity and survival rate of primary cultured cells, concentration of Aβ peptide in homogenates from various brain regions, the presence of neurofibrillary tangles and senile plaques in the brain, the total amyloid load in the brain, the density of synaptic terminals and the neuron counts in the brain. Additionally, behavioral analysis can be performed to measure learning and memory performance of the animals. The tests include, but are not limited to, the Morris water maze and the radial-arm maze. The measurements of biochemical and neuropathological parameters, and of behavioral parameters (learning and memory), are performed using methods well known to those skilled in the art. These transgenic, transplacement and knock-out animals can also be used to screen drugs that may influence these biochemical, neuropathological, and behavioral parameters relevant to AD. Cell lines can also be derived from these animals for use as cellular models of AD, or in drug screening.

Rational Drug Design

The goal of rational drug design is to produce structural analogs of biologically active polypeptides of interest or of small molecules with which they interact (e.g., agonists, antagonists, inhibitors) in order to fashion drugs which are, for example, more active or stable forms of the polypeptide, or which, e.g., enhance or interfere with the function of a polypeptide in vivo. Several approaches for use in rational drug design include analysis of three-dimensional structure, alanine scans, molecular modeling and use of anti-id antibodies. These techniques are well known to those skilled in the art.

Following identification of a substance which modulates or affects polypeptide activity, the substance may be further investigated. Furthermore, it may be manufactured and/or used in preparation, i.e., manufacture or formulation, or a composition such as a medicament, pharmaceutical composition or drug. These may be administered to individuals.

A substance identified as a modulator of polypeptide function may be peptide or non-peptide in nature. Non-peptide “small molecules” are often preferred for many in vivo pharmaceutical uses. Accordingly, a mimetic or mimic of the substance (particularly if a peptide) may be designed for pharmaceutical use.

The designing of mimetics to a known pharmaceutically active compound is a known approach to the development of pharmaceuticals based on a “lead” compound. This approach might be desirable where the active compound is difficult or expensive to synthesize or where it is unsuitable for a particular method of administration, e.g., pure peptides are unsuitable active agents for oral compositions as they tend to be quickly degraded by proteases in the alimentary canal. Mimetic design, synthesis and testing is generally used to avoid randomly screening large numbers of molecules for a target property.

Once the pharmacophore has been found, its structure is modeled according to its physical properties, e.g., stereochemistry, bonding, size and/or charge, using data from a range of sources, e.g., spectroscopic techniques, x-ray diffraction data and NMR. Computational analysis, similarity mapping (which models the charge and/or volume of a pharmacophore, rather than the bonding between atoms) and other techniques can be used in this modeling process.

A template molecule is then selected, onto which chemical groups that mimic the pharmacophore can be grafted. The template molecule and the chemical groups grafted thereon can be conveniently selected so that the mimetic is easy to synthesize, is likely to be pharmacologically acceptable, and does not degrade in vivo, while retaining the biological activity of the lead compound. Alternatively, where the mimetic is peptide-based, further stability can be achieved by cyclizing the peptide, increasing its rigidity. The mimetic or mimetics found by this approach can then be screened to see whether they have the target property, or to what extent it is exhibited. Further optimization or modification can then be carried out to arrive at one or more final mimetics for in vivo or clinical testing.

Diagnostic Assays

The identification of the interactions disclosed herein enables the development of diagnostic assays and kits, which can be used to determine a predisposition to or the existence of a physiological disorder. In one aspect, one of the proteins of the interaction is used to detect the presence of a “normal” second protein (i.e., normal with respect to its ability to interact with the first protein) in a cell extract or a biological fluid, and further, if desired, to detect the quantitative level of the second protein in the extract or biological fluid. The absence of the “normal” second protein would be indicative of a predisposition or existence of the physiological disorder. In a second aspect, an antibody against the protein complex is used to detect the presence and/or quantitative level of the protein complex. The absence of the protein complex would be indicative of a predisposition or existence of the physiological disorder.

### EXAMPLES

The present invention is further detailed in the following Examples, which are offered by way of illustration and are not intended to limit the invention in any manner. Standard techniques well known in the art or the techniques specifically described below are utilized.

### Example 1

### Yeast Two-hybrid System

The principles and methods of the yeast two-hybrid systems have been described in detail (Bartel and Fields, 1997). The following is thus a description of the particular procedure that was used, which was applied to all proteins.

The cDNA encoding the bait protein was generated by PCR from brain cDNA. Gene-specific primers were synthesized with appropriate tails added at their 5′ ends to allow recombination into the vector pGBTQ. The tail for the forward primer was 5′-GCAGGAAACAGCTATGACCATACAGTCAGCGGCCGCCACC-3′ (SEQ ID NO: 1) and the tail for the reverse primer was 5′-ACGGCCAGTCGCGTGGAGTGTTATGTCATGCGGCCGCTA-3′ (SEQ ID NO:2). The tailed PCR product was then introduced by recombination into the yeast expression vector pGBTQ, which is a close derivative of pGBTC (Bartel et al., 1996) in which the polylinker site has been modified to include M13 sequencing sites. The new construct was selected directly in the yeast J693 for its ability to drive tryptophane synthesis (genotype of this strain: Mat α, ade2, his3, leu2, trp1, URA3::GAL1-lacZ LYS2::GAL1-HIS3 gal4del gal80del cyhR2). In these yeast cells, the bait is produced as a C-terminal fusion protein with the DNA binding domain of the transcription factor Gal4 (amino acids 1 to 147). A total human brain (37 year-old male Caucasian) cDNA library cloned into the yeast expression vector pACT2 was purchased from Clontech (human brain MATCHMAKER cDNA, cat. # HL4004AH), transformed into the yeast strain J692 (genotype of this strain: Mat α, ade2, his3, leu2, trp1, URA3::GAL1-lacZ LYS2::GAL1-HIS3 gal4del gal80del cyhR2), and selected for the ability to drive leucine synthesis. In these yeast cells, each cDNA is expressed as a fusion protein with the transcription activation domain of the transcription factor Gal4 (amino acids 768 to 881) and a 9 amino acid hemagglutinin epitope tag. J693 cells (Mat α type) expressing the bait were then mated with J692 cells (Mat α type) expressing proteins from the brain library. The resulting diploid yeast cells expressing proteins interacting with the bait protein were selected for the ability to synthesize tryptophane, leucine, histidine, and β-galactosidase. DNA was prepared from each clone, transformed by electroporation into E. coli strain KC8 (Clontech KC8 electrocompetent cells, cat # C2023-1), and the cells were selected on ampicillin-containing plates in the absence of either tryptophane (selection for the bait plasmid) or leucine (selection for the brain library plasmid). DNA for both plasmids was prepared and sequenced by di-deoxynucleotide chain termination method. The identity of the bait cDNA insert was confirmed and the cDNA insert from the brain library plasmid was identified using BLAST program against public nucleotides and protein databases. Plasmids from the brain library (preys) were then individually transformed into yeast cells together with a plasmid driving the synthesis of lamin fused to the Gal4 DNA binding domain. Clones that gave a positive signal after β-galactosidase assay were considered false-positives and discarded. Plasmids for the remaining clones were transformed into yeast cells together with plasmid for the original bait. Clones that gave a positive signal after β-galactosidase assay were considered true positives.

### Example 2

### Identification of PS1-FKBP25 Interaction

A yeast two-hybrid system as described in Example 1 using amino acids 1-91 of PS1 (Swiss Protein (SP) accession No. P49768) as bait was performed. This PS1 fragment is the N-terminal cytostolic region. One clone that was identified by this procedure included amino acids 166-224 of FKBP25 (SP accession No. Q00688). FKBP25 has a rotamase domain in its C-terminal half, including the part that interacts with PS1.

### Example 3

### Identification of FKBP25-CIB Interaction

A yeast two-hybrid system as described in Example 1 using full length FKBP25 as bait was performed. One clone that was identified by this procedure included amino acids 1-191 of CIB (SP accession No. Q99828), a calcium binding protein.

### Example 4

### Identification of PS1-rab11 Interaction

A yeast two-hybrid system as described in Example 1 using amino acids 1-91 of PS1 as bait was performed. This PS1 fragment is the N-terminal cytostolic region. One clone that was identified by this procedure included amino acids 106-216 of rab11 (SP accession No. P24410). This portion of rab11 is the carboxy-tenninal region. This interaction is different than the interaction described in WO 97/27296, in which rab11 interacted with the TM6→7 loop domain.

### Example 5

### Identification of APP-BAT3 Interaction

A yeast two-hybrid system as described in Example 1 using amino acids 639-695 of APP (SP accession No. P05067) as bait was performed. This APP fragment is the C-terminal cytoplasmic fragment. One clone that was identified by this procedure included amino acids 603-1132 of BAT3 (SP accession No. P46379). This fragment of BAT3 includes the second proline-rich domain (amino acids 657-670).

### Example 6

### Identification of BAT3-6-adaptin Interaction

A yeast two-hybrid system as described in Example 1 using amino acids 1-241 of BAT3 as bait was performed. This APP fragment is the C-terminal cytoplasmic fragment. One clone that was identified by this procedure included amino acids 1062-1153 of δ-adaptin (GenBank (GB) accession No. AF002163).

### Example 7

### Identification of APP-PTPZ Interaction

A yeast two-hybrid system as described in Example 1 using amino acids 306-500 of APP695 as bait was performed. One clone that was identified by this procedure included amino acids 1052-1128 of PTPZ (SP accession No. P23471). This fragment of PTPZ is part of the extracellular domain (amino acids 25-1635).

### Example 8

### Identification of APP695-KIAA0351 Interaction

A yeast two-hybrid system as described in Example 1 using amino acids 306-500 of APP695 (GenBank (GB) accession no. Y00264; Swiss Protein (SP) accession no. P09000) as bait was performed. One clone that was identified by this procedure included amino acids 213-557 (C-terminus) of KIAA0351 (GB: AB002349).

### Example 9

### Identification of APP695-Prostaglandin D Synthase Interaction

A yeast two-hybrid system as described in Example 1 using amino acids 306-500 of APP695 (GB: Y00264; SP: P09000) as bait was performed. One clone that was identified by this procedure included amino acids 1-190 of prostaglandin D synthase (GB: M61900; SP: P412222).

### Example 10

### Identification of AChE-calpain Small Subunit Interaction

A yeast two-hybrid system as described in Example 1 using amino acids 31-136 of ACHE (GB: M55040; SP: P22303) as bait was performed. One clone that was identified by this procedure included amino acids 1-268 of calpain small (regulatory) subunit (GB: X04106; SP: P04632).

### Example 11

### Identification of AChE-KIAA0436 Interaction

A yeast two-hybrid system as described in Example 1 using amino acids 31-136 and 266-354 of AChE (GB: M55040; SP: P22303) as baits was performed. Clone that were identified by this procedure included amino acids 246-638 of KIAA0436 (GB: AB007896).

### Example 12

### Identification of AChE-α-Endosulfine Interaction

A yeast two-hybrid system as described in Example 1 using amino acids 31-136 of AChE (GB: M55040; SP: P22303) as bait was performed. One clone that was identified by this procedure included amino acids 24-121 of δ-endosulfine (GB: X99906).

### Example 13

### Identification of ACHE-GIPC Interaction

A yeast two-hybrid system as described in Example 1 using amino acids 31-136 of ACHE (GB: M55040; SP: P22303) as bait was performed. One clone that was identified by this procedure included amino acids 67-332 (C-terminus) of GIPC (GB: AF089816).

### Example 14

### Identification of AChE-δ-catenin Interaction

A yeast two-hybrid system as described in Example 1 using amino acids 63-534 and 355-517 of AChE (GB: M55040; SP: P22303) as baits was performed. Clones that were identified by this procedure included amino acids 689-1225 of δ-catenin (GB: U96136).

### Example 15

### Identification of δ-catenin-GIPC Interaction

A yeast two-hybrid system as described in Example 1 using amino acids 1006-1158 of δ-catenin (GB: U96136) as bait was performed. One clone that was identified by this procedure included amino acids 67-332 (C-terminus) of GIPC (GB: AF089816).

### Example 16

### Identification of δ-catenin-clathrin Interaction

A yeast two-hybrid system as described in Example 1 using amino acids 516-833 of δ-catenin (GB: U96136) as bait was performed. One clone that was identified by this procedure included amino acids 1311-1676 of the heavy chain of clathrin (GB: D21260; SP: Q00610).

### Example 17

### Identification of NACP-δ-catenin Interaction

A yeast two-hybrid system as described in Example 1 using amino acids 1-140 of NACP (GB: L00850; SP: P37840) as bait was performed. One clone that was identified by this procedure included amino acids 689-1225 of δ-catenin (GB: U96136).

### Example 18

### Identification of δ-catenin-Plakophilin 2 Interaction

A yeast two-hybrid system as described in Example 1 using amino acids 516-833 of δ-catenin (GB: U96136) as bait was performed. One clone that was identified by this procedure included amino acids 649-817 of plakophilin 2 (GB: X97675).

### Example 19

### Identification of ERAB-δ-catenin Interaction

A yeast two-hybrid system as described in Example 1 using amino acis 1-261 of ERAB (GB: U96132; SP: Q99714) as bait was performed. One clone that was identified by this procedure included amino acids 689-1225 of δ-catenin (GB: U96136).

### Example 20

### Identification of Bcl2-δ-catenin Interaction

A yeast two-hybrid system as described in Example 1 using amino acids 1-74 of Bcl2 (GB: M14745; SP: P10415) as bait was performed. One clone that was identified by this procedure included amino acids 689-1225 of δ-catenin (GB: U96136).

### Example 21

### Identification of δ-catenin-Bcr Interaction

A yeast two-hybrid system as described in Example 1 using amino acids 516-833 of δ-catenin (GB: U96136) as bait was performed. One clone that was identified by this procedure included amino acids 1100-1227 of Bcr (GB: U07000; SP: P11274).

### Example 22

### Identification of δ-catenin-14-3-3-beta Interaction

A yeast two-hybrid system as described in Example 1 using amino acids 1006-1158 of δ-catenin (GB: U96136) as bait was performed. One clone that was identified by this procedure included amino acids 1-245 of 14-3-3-beta (GB: X57346; SP: P31946).

### Example 23

### Identification of δ-catenin-14-3-3-zeta Interaction

A yeast two-hybrid system as described in Example 1 using amino acids and 1006-1158 of δ-catenin (GB: U96136) as bait was performed. One clone that was identified by this procedure included amino acids 1-245 of 14-3-3-zeta (GB: U28964; SP: P29213).

### Example 24

### Identification of δ-catenin-FAK2 Interaction

A yeast two-hybrid system as described in Example 1 using amino acids 1006-1158 of δ-catenin (GB: U96136) as bait was performed. One clone that was identified by this procedure included amino acids 625-1158 of FAK2 (GB: L49207; SP: Q13475).

### Example 25

### Identification of δ-catenin-Eps8 Interaction

A yeast two-hybrid system as described in Example 1 using amino acids 516-833 of δ-catenin (GB: U96136) as bait was performed. One clone that was identified by this procedure included amino acids 335-822 of Eps8 2 (GB: U12535; SP: Q12929).

### Example 26

### Identification of δ-catenin-KIAA0443 Interaction

A yeast two-hybrid system as described in Example 1 using amino acids 1006-1158 of δ-catenin (GB: U96136) as bait was performed. One clone that was identified by this procedure included amino acids 1161-1245 of KIAA0443 (GB: AB007903).

### Example 27

### Identification of PS-1-α-enolase Interaction

A yeast two-hybrid system as described in Example 1 using amino acids 1-91 of PS-1 (GB: L421110; SP: P49768) as bait was performed. One clone that was identified by this procedure included amino acids 135-433 of α-enolase (GB: AB007903).

### Example 28

### Identification of Axin-citrate Synthase Interaction

A yeast two-hybrid system as described in Example 1 using amino acids 301-600 of Axin (GB: AF009764) as bait was performed. One clone that was identified by this procedure included amino acids 1-123 of citrate synthase (GB: AF047042).

### Example 29

### Identification of Axin-aldolase C Interaction

A yeast two-hybrid system as described in Example 1 using amino acids 301-600 of Axin (GB: AF009764) as bait was performed. One clone that was identified by this procedure included amino acid residues of aldolase C (GB: AF054987; SP: P09972).

### Example 30

### Identification of Axin-creatine Kinase B Interaction

A yeast two-hybrid system as described in Example 1 using amino acids 1-300 of Axin (GB: AF009764) as bait was performed. One clone that was identified by this procedure included amino acids 252-381 of creatine kinase B (GB: L47647; SP: P12277).

### Example 31

### Identification of Axin-neurogranin Interaction

A yeast two-hybrid system as described in Example 1 using amino acids 301-600 of Axin (GB: AF009764) as bait was performed. One clone that was identified by this procedure included amino acids 1-78 of neurogranin (GB: U89165; SP: Q92686).

### Example 32

### Identification of Axin-Rab3A Interaction

A yeast two-hybrid system as described in Example 1 using amino acids 301-600 of Axin (GB: AF009764) as bait was performed. One clone that was identified by this procedure included amino acids 2-125 of Rab3A (GB: M28210; SP: P20336).

### Example 33

### Identification of Axin-AOP-1 Interaction

A yeast two-hybrid system as described in Example 1 using amino acids 301-600 and 451-750 of Axin (GB: AF009764) as baits was performed. Clones that were identified by this procedure included amino acids 1-256 of AOP-1 (GB: D49396; SP: P30048).

### Example 34

### Identification of Axin-SMN1 Interaction

A yeast two-hybrid system as described in Example 1 using amino acids 301-600 of Axin (GB: AF009764) as bait was performed. One clone that was identified by this procedure included amino acids 2-144 of SMN1 (GB: U18423; SP: Q16637).

### Example 35

### Identification of Axin-SRp30c Interaction

A yeast two-hybrid system as described in Example 1 using amino acids 301-600 of Axin (GB: AF009764) as bait was performed. One clone that was identified by this procedure included amino acids 175-221 of SRp30c (GB: U30825; SP: Q13242).

### Example 36

### Identification of PS-1-LSF Interaction

A yeast two-hybrid system as described in Example 1 using amino acids 1-91 of PS-1 (GB: L421110; SP: P49768) as bait was performed. One clone that was identified by this procedure included amino acids 405-502 of LSF (GB: U03494).

### Example 37

### Identification of LSF-APP Interaction

A yeast two-hybrid system as described in Example 1 using amino acids 393-502 of LSF (GB: U03494) as bait was performed. One clone that was identified by this procedure included amino acids 1-220 of APP (GB: Y00264; SP: P05067).

### Example 38

### Identification of LSF-4F5s Interaction

A yeast two-hybrid system as described in Example 1 using amino acids 393-502 of LSF (GB: U03494) as bait was performed. One clone that was identified by this procedure included amino acids 5-63 of 4F5s (GB: AF073518).

### Example 39

### Generation of Polyclonal Antibody against PS1-FKBP25 Complex

As shown above, APP interacts with FKBP25 to form a complex. A complex of the two proteins is prepared, e.g., by mixing purified preparations of each of the two proteins. If desired, the protein complex can be stabilized by cross-linking the proteins in the complex by methods known to those of skill in the art. The protein complex is used to immunize rabbits and mice using a procedure similar to the one described by Harlow et al. (1988). This procedure has been shown to generate Abs against various other proteins (for example, see Kraemer et al., 1993).

Briefly, purified protein complex is used as an immunogen in rabbits. Rabbits are immunized with 100 μg of the protein in complete Freund's adjuvant and boosted twice in three-week intervals, first with 100 μg of immunogen in incomplete Freund's adjuvant, and followed by 100 μg of immunogen in PBS. Antibody-containing serum is collected two weeks thereafter. The antisera is preadsorbed with APP and FKBP25, such that the remaining antisera comprises antibodies which bind conformational epitopes, i.e., complex-specific epitopes, present on the APP-FKBP25 complex but not on the monomers.

Polyclonal antibodies against each of the complexes set forth in Tables 1-37 are prepared in a similar manner by mixing the specified proteins together, immunizing an animal and isolating antibodies specific for the protein complex, but not for the individual proteins.

### Example 40

### Generation of Monoclonal Antibodies Specific for PS1-FKBP25 Complex

Monoclonal antibodies are generated according to the following protocol. Mice are immunized with immunogen comprising PS1-FKBP25 complexes conjugated to keyhole limpet hemocyanin using glutaraldehyde or EDC as is well known in the art. The complexes can be prepared as described in Example 39 may also be stabilized by crosslinking. The immunogen is mixed with an adjuvant. Each mouse receives four injections of 10 to 100 μg of immunogen, and after the fourth injection, blood samples are taken from the mice to determine if the serum contains antibodies to the immunogen. Serum titer is determined by ELISA or RIA. Mice with sera indicating the presence of antibody to the immunogen are selected for hybridoma production.

Spleens are removed from immune mice and a single-cell suspension is prepared (Harlow et al., 1988). Cell fusions are performed essentially as described by Kohler et al. (1975). Briefly, P3.65.3 myeloma cells (American Type Culture Collection, Rockville, Md.) or NS-1 myeloma cells are fused with immune spleen cells using polyethylene glycol as described by Harlow et al. (1988). Cells are plated at a density of 2×10⁵ cells/well in 96-well tissue culture plates. Individual wells are examined for growth, and the supernatants of wells with growth are tested for the presence of PS1-FKBP25 complex-specific antibodies by ELISA or RIA using PS1-FKBP25 complex as target protein. Cells in positive wells are expanded and subcloned to establish and confirm monoclonality.

Clones with the desired specificities are expanded and grown as ascites in mice or in a hollow fiber system to produce sufficient quantities of antibodies for characterization and assay development. Antibodies are tested for binding to PS1 alone or to FKBP25 alone, to determine which are specific for the PS1-FKBP25 complex as opposed to those that bind to the individual proteins.

Monoclonal antibodies against each of the complexes set forth in Tables 1-37 are prepared in a similar manner by mixing the specified proteins together, immunizing an animal, fusing spleen cells with myeloma cells and isolating clones which produce antibodies specific for the protein complex, but not for the individual proteins.

### Example 41

### In vitro Identification of Modulators for PS1-FKBP25 Interaction

The invention is useful in screening for agents, which modulate the interaction of PS1 and FKBP25. The knowledge that PS1 and FKBP25 form a complex is useful in designing such assays. Candidate agents are screened by mixing PS1 and FKBP25 (a) in the presence of a candidate agent and (b) in the absence of the candidate agent. The amount of complex formed is measured for each sample. An agent modulates the interaction of PS1 and FKBP25 if the amount of complex formed in the presence of the agent is greater than (promoting the interaction), or less than (inhibiting the interaction) the amount of complex formed in the absence of the agent. The amount of complex is measured by a binding assay that shows the formation of the complex, or by using antibodies immunoreactive to the complex.

Briefly, a binding assay is performed in which immobilized PS1 is used to bind labeled FKBP25. The labeled FKBP25 is contacted with the immobilized PS1 under aqueous conditions that permit specific binding of the two proteins to form an PS1-FKBP25 complex in the absence of an added test agent. Particular aqueous conditions may be selected according to conventional methods. Any reaction condition can be used, as long as specific binding of PS1-FKBP25 occurs in the control reaction. A parallel binding assay is performed in which the test agent is added to the reaction mixture. The amount of labeled FKBP25 bound to the immobilized PS1 is determined for the reactions in the absence or presence of the test agent. If the amount of bound, labeled FKBP25 in the presence of the test agent is different than the amount of bound labeled FKBP25 in the absence of the test agent, the test agent is a modulator of the interaction of PS1 and FKBP25.

Candidate agents for modulating the interaction of each of the protein complexes set forth in Tables 1-37 are screened in vitro in a similar manner.

### Example 42

### In vivo Identification of Modulators for PS1-FKBP25 Interaction

In addition to the in vitro method described in Example 41, an in vivo assay can also be used to screen for agents that modulate the interaction of PS1 and FKBP25. Briefly, a yeast two-hybrid system is used in which the yeast cells express (1) a first fusion protein comprising PS1 or a fragment thereof and a first transcriptional regulatory protein sequence, e.g., GAL4 activation domain, (2) a second fusion protein comprising FKBP25 or a fragment thereof and a second transcriptional regulatory protein sequence, e.g., GAL4 DNA-binding domain, and (3) a reporter gene, e.g., β-galactosidase, which is transcribed when an intermolecular complex comprising the first fusion protein and the second fusion protein is formed. Parallel reactions are performed in the absence of a test agent as the control and in the presence of the test agent. A functional PS1-FKBP25 complex is detected by detecting the amount of reporter gene expressed. If the amount of reporter gene expression in the presence of the test agent is different than the amount of reporter gene expression in the absence of the test agent, the test agent is a modulator of the interaction of PS1 and FKBP25.

Candidate agents for modulating the interaction of each of the protein complexes set forth in Tables 1-37 are screened in vivo in a similar manner.

While the invention has been disclosed in this patent application by reference to the details of preferred embodiments of the invention, it is to be understood that the disclosure is intended in an illustrative rather than in a limiting sense, as it is contemplated that modifications will readily occur to those skilled in the art, within the spirit of the invention and the scope of the appended claims.

### LIST OF REFERENCES

Abe, K. and Kimura, H. (1996). J Neurochem 67:2074-2078.

Aguilar-Bryan, L. et al. (1995). Science 268:423-426.

Ahnert-Hilger, G. et al. (1996). Eur J CellBiol 70:1-11.

Aitken, A. et al. (1995). Mol Cell Biochem 149-150:41-49.

Albert, M. S. (1996). Proc Natl Acad Sci USA 93:13547-13551.

Alvarez, A. et al. (1998). J Neurosci 18:3213-3223.

Annaert, W. G. et al. (1999). J Cell Biol 147:277-294.

Araki, W. et al. (1991). Biochem Biophys Res Commun 181:265-271.

Aronheim et al., (1997). Mol. Cell. Biol. 17:3094-3102.

Ashall, F. and Goate, A. M. (1994). Trends Biochem Sci 19:42-46.

Ashcroft, F. M. (1988). Annu Rev Neurosci 11:97-118.

Avraham, S. et al. (1995). J Biol Chem 270:27742-27751.

Bancher, C. et al. (1997). J Neural Transm 104 Suppl. 50:141-152.

Banerji, J. et al. (1990). Proc Natl Acad Sci USA 87:2374-2378.

Barger, S. W. et al. (1995). J Neurochem 64:2087-2096.

Barger, S. W. and Mattson, M. P. (1995). Biochem J 311:45-47.

Barger, S. W. and Mattson, M. P. (1996). Brain Res Mol Brain Res 40:116-126.

Bartel, P. L. et al. (1993). “Using the 2-hybrid system to detect protein-protein interactions.” In: Cellular Interactions in Development: A Practical Approach, Oxford University Press, pp. 153-179.

Bartel, P. L. et al. (1996). Nat Genet 12:72-77.

Bartel, P. L. and Fields, S. (1997). The Yeast Two-Hybrid System. New York: Oxford University Press.

Beal, M. F. (1996). Curr Opin Neurobiol 6:661-666.

Beal, M. F. (1998). Biochim Biophys Acta 1366:211-223.

Bechade, C. et al. (1999). Eur J Neurosci 11:293-304.

Behl, C. et al. (1992). Biochem Biophys Res Commun 186:944-950.

Behl, C. et al. (1994). Brain Res 645:253-264.

Bertram, P. G. et al. (1998). Curr Biol 8:1259-1267.

Beyreuther, K. et al. (1996). Ann NY Acad Sci 777:74-76.

Blanco, G. et al. (1998). Mamm Genome 9:473-475.

Borchelt, D. R. et al. (1996). Neuron 17:1005-1013.

Bowes, M. P. et al. (1994). Exp Neurol 129:112-119.

Braselmann, S. and McCormick, F. (1995). EMBO J 14:4839-4848.

Breen, K. C. et al. (1991). J Neurosci Res 28:90-100.

Brion, J. P. (1998). Acta Neurol Belg 98:165-174.

Bryan, J. et al. (1997). Curr Opin Cell Biol 9:553-559.

Busciglio, J. and Yankner, B. A. (1995). Nature 378:776-779.

Cameron, A. M. et al. (1995). Cell 83:463-472.

Caputi, A. et al. (1997). J Neurochem 68:2523-2529.

Castillo, P. E. et al. (1997). Nature 388:590-593.

Chen, W. et al. (1998). Mol Biol Cell 9:3241-3257.

Chevray, P. M. and Nathans, D. N. (1992). Proc. Natl. Acad. Sci. USA 89:5789-5793.

Chui, D. H. et al. (1999). Nat Med 5:560-564.

Citron, M. et al. (1998). Neurobiol Dis 5:107-116.

Cribbs, D. H. et al. (1994). Soc Neurosci Abstr 20:604.

Cruts, M. and Van Broeckhoven, C. (1998). Hum Mutat 11:183-190.

Cummings, J. L. et al. (1998). Neurology 51:S2-17.

Davis, S. and Laroche, S. (1998). C R Acad Sci III321:97-107.

Dell'Angelica, E. C. et al. (1998). Science 280:431-434.

Deretic, D. (1997). Electrophoresis 18:2537-2541.

De Strooper, B. et al (1999). Nature 398:518-522.

De Vries, L. et al. (1998a). Mol Biol Cell 9:1123-1134.

De Vries, L. et al. (1998b). Proc Natl Acad Sci USA 95:12340-12345.

Denny, J. B. et al. (1990). Brain Res 534:317-320.

Dickson, D. W. (1997). J Neuropathol Exp Neurol 56:321-339.

Diekmann, D. et al. (1995). EMBO J 14:5297-5305.

Dierick, H. and Bejsovec, A. (1999). Curr Top Dev Biol 43:153-190.

Doan, A. et al. (1996). Neuron 17:1023-1030.

Doyle, E. et al. (1990). Neurosci Lett 115:97-102.

Duff, K. et al (1996). Nature 383:710-713.

Efthimiopoulos, S. et al. (1998). J Neurochem 71:2365-2372.

Ernekova, K. S. et al. (1997). J Biol Chem 272:32869-32877.

Fagarasan, M. O. and Aisen, P. S. (1996). Brain Res 723:231-234.

Falduto, M. T. and LaDu, M. J. (1996). The role of apolipoprotein E in neurobiology and Alzheimer's Disease. In: Alzheimer's Disease (Brioni J D, Decker M W eds), pp. New York: Wiley Press.

Fazioli, F. et al. (1993). EMBO J12:3799-3808.

Fields, S. and Song, O-K. (1989). Nature 340:245-246.

Freedman, J. E. and Lin, Y-J. (1996). Neuroscientist 2:145-152.

Freeman, K. and Livi, G. P. (1996). Gene 172:143-147.

Furukawa, K. et al. (1996a). Nature 379:74-78.

Furukawa, K. et al. (1996b). J Neurochem 67:1882-1896.

Galat, A. et al. (1992). Biochemistry 31:2427-2434.

Geppert, M. and Sudhof, T. C. (1998). Annu Rev Neurosci 21:75-95.

Gerendasy, D. D. and Sutcliffe, J. G. (1997). Mol Neurobiol 15:131-163.

Gillardon, F. et al. (1996). Brain Res 706:169-172.

Girault, J-A. et al. (1990). J Neurosci 10:1124-1133.

Gooch, M. D. and Stennett, D. J. (1996). Am J Health Syst Pharm 53:1545-1557.

Goodman, Y. and Mattson, M. P. (1994). Exp Neurol 128:1-12.

Govoni, S. et al. (1996). Ann NY Acad Sci 777:332-337.

Gromov, P. S. et al. (1998). FEBS Lett 429:359-364.

Gschwind, M. et al. (1996). Ann NY Acad Sci 777:293-296.

Guo, Q. et al. (1996). Neuroreport 8:379-383.

Guo, Q. et al. (1997). J Neurosci 17:4212-4222.

Guo, Q. et al. (1998). J Biol Chem 273:12341-12351.

Guo, Q. et al. (1999a). J Neurosci Res 56:457-470.

Guo, Q. et al. (1999b). Nat Med 5:101-106.

Haass, C. and De Strooper, B. (1999). Science 286:916-919.

Hardy, J. (1995). Am J Med Genet 60:456-460.

Hardy, J. (1997). Trends Neurosci 20:154-159.

Hardy, J. and Gwinn-Hardy, K. (1998). Science 282:1075-1079.

Harlow et al. (1988). Antibodies: A Laboratory Manual (Cold Spring Harbor Laboratory, Cold Spring Harbor, N.Y.).

Hatzfeld, M. (1999). Int Rev Cytol 186:179-224.

He, X. Y. et al. (1998). J Biol Chem 273:10741-10746.

Hirst, J. and Robinson, M. S. (1998). Biochim Biophys Acta 1404:173-193.

Hockenbery, D. et al. (1990). Nature 348:334-336.

Horiuchi, A. et al. (1990). J Biol Chem 265:9476-9484.

Huber, G. et al. (1993). Brain Res 603:348-352.

Huber, G. et al. (1997). Neuroscience 80:313-320.

Hubberstey, A. et al. (1996). J Cell Biochem 61:459-466.

Hung, D. T. and Schreiber, S. L. (1992). Biochem Biophys Res Commun 184:733-738.

Hussain, I. et al. (1999). Mol Cell Neurosci in press:

Hutton, M. and Hardy, J. (1997). Hum Mol Genet 6:1639-1646.

Ii, K. (1995). Drugs Aging 7:97-109.

Inagaki, N. et al. (1995). Science 270:1166-1170.

Inagaki, N. et al. (1997). FEBS Lett 409:232-236.

Inestrosa, N. C. and Alarcon, R. (1998). J Physiol Paris 92:341-344.

Ishii, K. et al. (1997). Neurosci Lett 228:17-20.

Ishikawa, K. et al. (1997). DNA Res 4:307-313.

Iversen, L. L. et al. (1995). Biochem J311:1-16.

Iwai, A. et al. (1995a). Neuron 14:467-475.

Iwai, A. et al. (1995b). Biochemistry 34:10139-10145.

Jensen, P. H. et al. (1998). J Biol Chem 273:26292-26294.

Jin, L-W. et al. (1994). Proc Natl Acad Sci USA 91:5461-5470.

Jin, L-W. and Saitoh, T. (1995). Drugs Aging 6:136-149.

Jin, Y. J. et al. (1992). J Biol Chem 267:10942-10945.

Jin, Y. J. and Burakoff, S. J. (1993). Proc Natl Acad Sci USA 90:7769-7773.

Johannes, L. et al. (1994). EMBO J13:2029-2037.

Kalaria, R. N. et al. (1996a). Neurodegeneration 5:497-503.

Kalaria, R. N. et al. (1996b). Neurobiol Aging 17:687-693.

Kim, S. S. et al. (1997). J Mol Neurosci 9:49-54.

Kim, T. W. and Tanzi, R. E. (1997). Curr Opin Neurobiol 7:683-688.

Kimura, S. et al. (1998). J Neurosci 18:8551-8558.

Kitamura, Y. et al. (1999). Biochem Biophys Res Commun 254:582-586.

Kohler, G. and Milstein, C. (1975). Nature 256, 495-497.

Komori, N. et al. (1997). Mol Brain Res 49:103-112.

Koo, E. H. et al. (1990). Proc Natl Acad Sci USA 87:1561-1565.

Kosik, K. S. (1998). Science 279:463-465.

Kosik, K. S. (1999). Nat Med 5:149-150.

Kovacs, D. M. and Tanzi, R. E. (1998). Cell Mol Life Sci 54:902-909.

Kraemer, F. B. et al. (1993). J Lipid Res. 34, 663-672.

Krueger, N. X. and Saito, H. (1992). Proc Natl Acad Sci USA 89:7417-7421.

Kruman, I. et al. (1997). J Neurosci 17:5089-5100.

Lai, F. et al. (1994). Genomics 22:610-616.

Leanna, C. A. and Hannink, M. (1996). Nucl. Acids Res. 24:3341-3347.

Leblanc, A. C. et al. (1996). J Neurochem 66:2300-2310.

Le Borgne, R. et al. (1998). J Biol Chem 273:29451-29461.

Lee, R. K. et al. (1999). J Neurosci 19:940-947.

Lefebvre, S. et al. (1995). Cell 80:155-165.

Lefebvre, S. et al. (1998). Hum Mol Genet 7:1531-1536.

Lehmann, S. et al. (1997). J Biol Chem 272:12047-12051.

Leimer, U. et al. (1999). Biochemistry 38:13602-13609.

Lemere, C. A. et al. (1996). Nature Med 2:1146-1150.

Levesque, G. et al. (1999). J Neurochem 72:999-1008.

Li, H. L. et al. (1997). J Neurobiol 32:469-480.

Li, J. et al. (1998). Brain Res Mol Brain Res 60:77-88.

Li, Q. X. et al. (1995). J Biol Chem 270:14140-14147.

Lippa, C. F. (1999). Int J Mol Med 4:529-536.

Loo, D. T. et al. (1993). Proc Natl Acad Sci USA 90:7951-7955.

Lorenz, M. C. and Heitman, J. (1995). J Biol Chem 270:27531-27537.

Ma, J. Y. et al. (1996). Neurobiol Aging 17:773-780.

Manelli, A. M. and Puttfarcken, P. S. (1995). Brain Res Bull 38:569-576.

Mark, R. J. et al. (1995). J Neurosci 15:6239-6249.

Mark, R. J. et al. (1997). J Neurochem 68:255-264.

Masliah, E. et al. (1996). Am J Pathol 148:201-210.

Masliah, E. et al. (1997). Neurosci 78:135-146.

Mattson, M. P. et al. (1993a). Trends Neurosci 16:409-414.

Mattson, M. P. et al. (1993b). Neuron 10:243-254.

Mattson, M. P. et al. (1995). J Neurochem 65:1740-1751.

Mattson M P (1997a). AlzDis Review 2:1-14.

Mattson M P (1997b). Neurosci Biobehav Rev 21:193-206.

Mattson, M. P. (1997c). Physiol Rev 77:1081-1132.

Mattson, M. P. et al. (1999). Soc Neurosci Abstr 25:1600.

Matzel, L. D. et al. (1998). Rev Neurosci 9:129-167.

McLoughlin, D. M. and Miller, C. C. J. (1996). FEBS Lett 397:197-200.

McRae, A. et al. (1997). Gerontology 43:95-108.

Mertens, C. et al. (1996). J Cell Biol 135:1009-1025.

Meziane, H. at al. (1998). Proc Natl Acad Sci USA 95:12683-12688.

Milward, E. A. et al (1992). Neuron 9:129-137.

Mook-Jung, I. and Saitoh, T. (1997). Neurosci Lett 235:1-4. Erratum: Neurosci Lett 239:131.

Mucke, L. et al. (1995). J Exp Med 181:1551-1556.

Muller, D. et al. (1995). Synapse 19:37-45.

Murayama, M. et al. (1998). FEBS Lett 433:73-77.

Nagy, Z. et al. (1999). Acta Neuropathol 97:346-354.

Naik, U. P. et al. (1997). J Biol Chem 272:4651-4654.

Nakamura, T. et al (1998). Genes Cells 3:395-403.

Nalbantoglu, J. et al. (1997). Nature 387:500-505.

Neve, R. L. et al. (1990). Prog Brain Res 86:257-267.

Ninomiya, H. et al (1993). J Cell Biol 121:879-886.

Ninomiya, H. et al. (1994). J Neurochem 63:495-500.

Nixon, R. A. et al. (1994). Ann NY Acad Sci 747:77-91.

Octave, J. N. (1995). Rev Neurosci 6:287-316.

Olesen, O. F. et al. (1997). Brain Res Mol Brain Res 44:105-112.

Oyama, F. et al. (1998). J Neurochem 71:313-322.

Park, J. B. et al. (1997). J Biol Chem 272:20857-20865.

Parsons, J. T. et al. (1994). J Cell Sci 18:109-113.

Pasinetti, G. M. (1996). Neurobiol Aging 17:707-716.

Pasineffi, G. M. and Aisen, P. S. (1998). Neuroscience 87:319-324.

Pellizzoni, L. et al. (1998). Cell 95:615-624.

Pendergast, A. M. et al. (1991). Cell 66:161-171.

Pietrzik, C. U. et al. (1998). Proc Natl Acad Sci USA 95:1770-1775.

Polymeropoulos, M. H. et al. (1997). Science 276:2045-2047.

Prasad, K. N. et al. (1998). Proc Soc Exp Biol Med 219:120-125.

Price, D. L., et al. (1995). Curr Opin Neurol 8:268-274.

Provenzano, C. et al. (1998). Exp Cell Res 242:186-200.

Rapoport, S. I. et al. (1996). Neurodegeneration 5:473-476.

Ray, W. J. et al., Ashall F, Goate A M (1998). Mol Med Today 4:151-157.

Renbaum, P. and Levy-Lahad, E. (1998). Cell Mol Life Sci 54:910-919.

Richardson, J. S. et al. (1996). Ann NY Acad Sci 777:362-367.

Roch, J-M. and Puttfarcken, P. S. (1996). Alz ID Res Al 1:9-16.

Roch, J-M. et al. (1992). J Biol Chem 267:2214-2221.

Roch, J-M. et al. (1993). Ann NY Acad Sci 695:149-157.

Roch, J-M. et al. (1994). Proc Natl Acad Sci USA 91:7450-7454.

Roch, J-M. et al. (1997). Soc Neurosci Abstr 23:855.

Rossner, S. et al. (1998). Prog Neurobiol 56:541-569.

Russo, T. et al. (1998). FEBS Lett 434:1-7.

Sabo, S. L. et al. (1999). J Biol Chem 274:7952-7957.

Sah, P. and Bekkers, J. M. (1996). J Neurosci 16:4537-4542.

Saito, K. et al. (1993). Proc Natl Acad Sci USA 90:2628-2632.

Saitoh, T. et al. (1989). Cell 58:615-622.

Saitoh, T. et al. (1991). Lab Invest 64:596-616.

Saitoh, T. et al. (1994). The Biological Function of Amyloid β/A4 Protein Precursor. In: Amyloid Protein Precursor in Development, Aging, and Alzheimer's Disease (Masters, C. L. et al., eds), pp 90-99. Berlin: Springer-Verlag.

Saitoh, T. et al. (1995). Induction of Signal-transducing Pathways by APP Binding to a Cell Surface Receptor. In: Research Advances in Alzheimer's Disease and Related Disorders (Iqbal, J. A. et al. eds), pp 693-699. New York: John Wiley & Sons Ltd.

Saitoh, T. and Roch, J-M. (1995). DN&P 8:206-215.

Sasaki, H. et al. (1995). J Biol Chem 270:21206-21219.

Schaller, M. D. (1997). Soc Gen Physiol Ser 52:241-255.

Schaller, M. D. and Parsons, J. T. (1994). Curr Opin Cell Biol 6:705-710.

Scharf, J. M. et al. (1998). Nat Genet 20:83-86.

Schrader-Fischer, G. et al. (1997). J Neurochem 68:1571-1580.

Schubert, D. (1997). Eur J Neurosci 9:770-777.

Screaton, G. R. et al. (1995). EMBO J14:4336-4349.

Selkoe, D. J. (1994a). J Neuropathol Exp Neurol 53:438-447.

Selkoe, D. J. (1994b). Annu Rev Neurosci 17:489-517.

Selkoe, D. J. (1994c). Annu Rev Cell Biol 10:373-403.

Selkoe, D. J. (1996a). J Biol Chem 271:18295-18298.

Selkoe, D. J. (1996b). Cold Spring Harb Symp Quant Biol 61:587-596.

Selkoe, D. J. et al. (1996c). Ann NY Acad Sci 777:57-64.

Selkoe, D. J. (1997). Science 275:630-631.

Selkoe, D. J. (1998). Trends Cell Biol 8:447-453.

Selkoe, D. J. (1999). Nature 399:A23-A31.

Shapiro, I. P. et al. (1991). J Neurochem 56:1154-1162.

Sheehan, D. et al. (1996). Neuroreport 7:1297-1300.

Sheng, J. G. et al. (1997). Acta Neuropathol (Berl) 94:1-5.

Shinozaki, K. et al. (1998). Int J Mol Med 1:797-799.

Shintani, T. et al. (1998). Neurosci Lett 247:135-138.

Siciliano, J. C. et al. (1996). J Biol Chem 271:28942-28946.

Simonian, N. A. and Coyle, J. T. (1996). Annu Rev Pharmacol Toxicol 36:83-106.

Sinha, S. and Lieberburg, I. (1999). Proc Natl Acad Sci USA 96:11049-11053.

Skoulakis, E. M. and Davis, R. L. (1998). Mol Neurobiol 16:269-284.

Small, D. H. et al. (1994). J Neurosci 14:2117-2127.

Smith-Swintosky, V. L. et al. (1994). J Neurochem 63:781-784.

Snyder, S. H. et al. (1998). Trends Pharmacol Sci 19:21-26.

Spies, T. et al. (1989a). Science 243:214-217.

Spies, T. et al. (1989b). Proc Natl Acad Sci USA 86:8955-8958.

Stabler, S. M. et al. (1999). J Cell Biol 145:1277-1292.

Stahl, B. et al. (1999). J Biol Chem 274:9141-9148.

Steiner, H. et al. (1998). J Biol Chem 273:32322-32331.

Steiner, H. et al. (1999). J Biol Chem 274:28669-28673.

Steiner, J. P. et al. (1997a). Nat Med 3:421-428.

Steiner, J. P. et al. (1997b). Proc Natl Acad Sci USA 94:2019-2024.

Strittmatter, W. J. and Roses, A. D. (1995). Proc Natl Acad Sci USA 92:4725-4727.

Storey, E. and Cappai, R. (1999). Neuropathol Appl Neurobiol 25:81-97.

Storm, D. R. et al. (1998). Neuron 20:1199-1210.

Suzuki, K. et al. (1995). Biol Chem Hoppe Seyler 376:523-529.

Talbot, K. et al. (1997). Hum Mol Genet 6:497-500.

Tanahashi, H. and Tabira, T. (1999). Neuroreport 10:563-568.

Tesco, G. et al. (1998). J Biol Chem 273:33909-33914.

Thinakaran, G. et al. (1998). Neurobiol Dis 4:438-453.

Tomita, T. et al. (1997). Proc Natl Acad Sci USA 94:2025-2030.

Trommsdorff M, Borg J P, Margolis B, Herz J (1998). J Biol Chem 273:33556-33560.

Ueda, K. et al. (1993). Proc Natl Acad Sci USA 90:11282-11286.

Ullrich, 0. et al. (1996). J Cell Biol 135:913-924.

Urbe, S. et al. (1993). FEBS Lett 334:175-182.

Vassar, R. et al. (1999). Science 286:735-741.

Villacres, E. C. et al. (1998). J Neurosci 18:3186-3194.

Virsolvy-Vergine, A. et al. (1996). Diabetologia 39:135-141.

Vito, P. et al. (1996). J Biol Chem 271:31025-31028.

Wallace, W. C. et al. (1997a). Brain Res Mol Brain Res 52:201-212.

Wallace, W. C. et al. (1997b). Brain Res Mol Brain Res 52:213-227.

Wang, R. and Liew, C. C. (1994). Mol Cell Biochem 136:49-57.

Warmuth, M. et al. (1999). Ann Hematol 78:49-64.

Weidemann, A. et al. (1997). Nature Med 3:328-332.

Weiss, J. H. et al (1994). J Neurochem 62:372-375.

Wiederrecht, G. et al. (1992). Biochem Biophys Res Commun 185:298-303.

Wolfe, M. S. et al. (1999a). Biochemistry38:11223-11230.

Wolfe, M. S. et al. (1999b). Nature 398:513-517.

Wolozin, B. et al. (1996). Science 274:1710-1713.

Wong, W. T. et al. (1994). Oncogene 9:3057-3061.

Xia, W. et al. (1998). Biochemistry 37:16465-16471.

Yamada, M. et al. (1996). Stroke 27:1155-1162.

Yamamoto, K. et al. (1994). J Neurobiol 25:585-594.

Yan, S. D. et al. (1999). J Biol Chem 274:2145-2156.

Yoshimoto, M. et al. (1995). Proc Natl Acad Sci USA 92:9141-9145.

Yu, G. et al. (1998). J Biol Chem 273:16470-16475.

Yu, H. et al. (1996). J Biol Chem 271:29993-29998.

Zambrano, N. et al. (1998). J Biol Chem 273:20128-20133.

Zelicof, A. et al. (1996). J Biol Chem 271:18243-18252.

Zhang, Z. et al. (1998). Nature 395:698-702.

Zhou, J. H. et al. (1997a). Neuroreport 8:2085-2090.

Zhou, J. H. et al. (1997b). Neuroreport 8:1489-1494

PCT Published Application No. WO 97/27296

U.S. Pat. No. 5,622,852

U.S. Pat. No. 5,773,218

What is claimed is:

1. An isolated protein complex comprising two proteins, the protein complex selected from the group consisting of:
(a) a complex of acetylcholine esterase and KIAA0436;
(b) a complex of a fragment of acetylcholine esterase and KIAA0436;
(c) a complex acetylcholine esterase and a fragment of KIAA0436; and
(d) a complex of a fragment of acetylcholine esterase and a fragment of KIAA0436.

2. The protein complex of claim 1, wherein said protein complex comprises acetylcholine esterase and KIAA0436.

3. The protein complex of claim 1, wherein said protein complex comprises a fragment of acetylcholine esterase and KIAA0436 or acetylcholine esterase and a fragment of KIAA0436.

4. The protein complex of claim 1, wherein said protein complex comprises fragments of acetylcholine esterase and KIAA0436.

5. An isolated antibody selectively immunoreactive with a protein complex of claim 1.

6. The antibody of claim 5, wherein said antibody is a monoclonal antibody.

7. A method for diagnosing a neurodegenerative disorder in an animal, which comprises assaying for:
(a) whether a protein complex set forth in claim 1 is present in a tissue extract;
(b) the ability of proteins to form a protein complex set forth in claim 1; and
(c) a mutation in a gene encoding a protein of a protein complex set forth in claim 1.

8. The method of claim 7, wherein said animal is a human.

9. The method of claim 8, wherein said neurodegenerative disorder is demential.

10. The method of claim 8, wherein said neurodegenerative disorder is Alzheimer's Disease.

11. The method of claim 7, wherein the diagnosis is for a predisposition to said neurodegenerative disorder.

12. The method of claim 7, wherein the diagnosis is for the existence of said neurodegenerative disorder.

13. The method of claim 7, wherein said neurodegenerative disorder is dementia.

14. The method of claim 7, wherein said neurodegenerative disorder is Alzheimer's Disease.

15. The method of claim 7, wherein said assay comprises a yeast two-hybrid assay.

16. The method of claim 7, wherein said assay comprises measuring in vitro a complex formed by combining the proteins of the protein complex, said proteins isolated from said animal.

17. The method of claim 16, wherein said complex is measured by binding with an antibody specific for said complex.

18. The method of claim 7, wherein said assay comprises mixing an antibody specific for said protein complex with a tissue extract from said animal and measuring the binding of said antibody.

19. A method for determining whether a mutation in a gene encoding one of the proteins of a protein complex set forth in claim 1 is useful for diagnosing a neurodegenerative disorder, which comprises assaying for the ability of said protein with said mutation to form a complex with the other protein of said protein complex, wherein an inability to form said complex is indicative of said mutation being useful for diagnosing a neurodegenerative disorder.

20. The method of claim 19, wherein said gene is an animal gene.

21. The method of claim 20, wherein said animal is a human.

22. The method of claim 21, wherein said neurodegenerative disorder is dementia.

23. The method of claim 21, wherein said neurodegenerative disorder is Alzheimer' Disease.

24. The method of claim 19, wherein the diagnosis is for a predisposition to a neurodegenerative disorder.

25. The method of claim 19, wherein the diagnosis is for the existence of a neurodegenerative disorder.

26. The method of claim 19, wherein said assay comprises a yeast two-hybrid assay.

27. The method of claim 19, wherein said assay comprises measuring in vitro a complex formed by combining the proteins of the protein complex, said proteins isolated from an animal.

28. The method of claim 27, wherein said animal is a human.

29. The method of claim 27, wherein said complex is measured by binding with an antibody specific for said complex.

30. A non-human animal model for a neurodegenerative disorder wherein the genome of said animal or an ancestor thereof has been modified such that the formation of a protein complex set forth in claim 1 has been altered.

31. The non-human animal model of claim 30, wherein said neurodegenerative disorder is demential.

32. The non-human animal model of claim 30, wherein said neurodegenerative disorder is Alzheimer's Disease.

33. The non-human anirmal model of claim 30, wherein the formation of said protein complex has been altered as a result of:
(a) over-expression of at least one of the proteins of said protein complex;
(b) replacement of a gene for at least one of the proteins of said protein complex with a gene from a second animal and expression of said protein;
(c) expression of a mutant form of at least one of the proteins of said protein complex;
(d) a lack of expression of at least one of the proteins of said protein complex; or
(e) reduced expression of at least one of the proteins of said protein complex.

34. A cell line obtained from the animal model of claim 30.

35. A non-human animal model for a neurodegenerative disorder, wherein the biological activity of a protein complex set forth in claim 1 has been altered.

36. The non-human animal model of claim 35, wherein said neurodegenerative disorder is demential.

37. The non-human animal model of claim 35, wherein said neurodegenerative disorder is Alzheimer's Disease.

38. The non-human animal model of claim 35, wherein said biological activity has been altered as a result of:
(a) disrupting the formation of said complex; or
(b) disrupting the action of said complex.

39. The non-human animal model of claim 35, wherein the formation of said complex is disrupted by binding an antibody to at least one of the proteins which form said protein complex.

40. The non-human animal model of claim 35, wherein the action of said complex is disrupted by binding an antibody to said complex.

41. The non-human animal model of claim 35, wherein the formation of said complex is disrupted by binding a small molecule to at least one of the proteins which form said protein complex.

42. The non-human animal model of claim 35, wherein the action of said complex is disrupted by binding a small molecule to said complex.

43. A cell in which the genome of cells of said cell line has been modified to produce at least one protein complex set forth in claim 1.

44. A cell line in which the genome of the cells of said cell line has been modified to eliminate at least one protein of a protein complex set forth in claim 1.

45. A composition comprising:
a first expression vector having a nucleic acid encoding acetylcholine esterase or a homologue or derivative or fragment thereof, and
a second expression vector having a nucleic acid encoding KIAA0436, or a homologue or derivative or fragment thereof.

46. A host cell comprising:
a first expression vector having a nucleic acid encoding a first protein which is acetylcholine esterase or a homologue or derivative or fragment thereof, and
a second expression vector having a nucleic acid encoding a second protein which is KIAA0436, or a homologue or derivative or fragment thereof.

47. The host cell of claim 46, wherein said host cell is a yeast cell.

48. The host cell of claim 46, wherein said first and second proteins are expressed in fusion proteins.

49. The host cell of claim 46, wherein one of said first and second nucleic acids is linked to a nucleic acid encoding a DNA binding domain, and the other of said first and second nucleic acids is linked to a nucleic acid encoding a transcription-activation domain, whereby two fusion proteins can be produced in said host cell.

50. The host cell of claim 46, further comprising a reporter gene, wherein the expression of the reporter gene is determined by the interaction between the first polypeptide and the second polypeptide.
